(19) United States
(12) Patent Application Publication
(10) Pub. No.: US 2021/0147601 A1
(43) Pub. Date: May 20, 2021

(54) BENZOXAZINE BLENDS

(71) Applicant: WESTERN WASHINGTON UNIVERSITY, Bellingham, WA (US)

(72) Inventors: David Allen Rider, White Rock (CA); Emily Ann Brown, Bellingham, WA (US); Ayesha Nadeem, Bellingham, WA (US)

(73) Assignee: WESTERN WASHINGTON UNIVERSITY, Bellingham, WA (US)

(21) Appl. No.: 17/044,748

(22) PCT Filed: Apr. 1, 2019
(86) PCT No.: PCT/US2019/025186

§ 371 Date: Oct. 1, 2020

Related U.S. Application Data
(60) Provisional application No. 62/651,198, filed on Apr. 1, 2018.

(51) Int. Cl. C08F 283/06; C07D 413/06; C08K 7/14; C08K 7/06; C08K 3/04

(52) U.S. Cl. CPC C08F 283/06; C07D 413/06; C08K 2201/011; C08K 7/06; C08K 3/041; C08K 7/14

(57) ABSTRACT

Provided herein are blends of benzoxazine-containing monomers with polymer having one or more leaving groups, such as tosylated polymers (e.g., tosylated polyethylene glycol (PEG)); or blends of benzoxazine-containing monomers with particles/fibers having one or more leaving groups.

### CROSS-REFERENCE TO RELATED APPLICATION

This application claims the benefit of U.S. Patent Application No. 62/651,198, filed Apr. 1, 2018, the disclosure of which is hereby incorporated by reference in its entirety.

### BACKGROUND

Polybenzoxazines, PBA-a or P(BA-a)s, are an emerging class of thermosetting resins that display excellent properties such as high glass transition temperatures, low water absorption and flammability, near-zero volumetric cure shrinkage, and very good dielectric, thermal and mechanical properties even at modest polymer molecular weights. Crosslinked P(BA-a)s are typically produced from monomers containing two or more benzoxazine rings per molecule and the ring-opening polymerization (ROP) of these rings leads to a macromolecular networked structure mostly consisting of Mannich-bridged phenolic moieties. The synthetic scope for benzoxazine monomers, which usually consists of the condensation of formaldehyde with a primary amine and a phenolic molecule, is very large and has led to a wide class of P(BA-a)s. Recent examples include P(BA-a)s with hydroxyl, phenyl, maleimide, propargyl, allyl, and carboxy crosslinking groups, or aliphatic residues for tailored thermal stability, low-brittleness and other properties. The ROP is commonly triggered by a high temperature condition (from 160-270° C.). The high temperatures required for the thermal ROP of benzoxazines have in part contributed to the lack of widespread deployment of these resins into various industries. Accelerators such as organic acids and bases, Lewis acids, imidazoles, transition metal compounds, and metal-organic frameworks, can reduce this polymerization temperature.

Functionalizing PBA-a with other polymers offers interesting opportunities for creating hybrid materials with stable, tuned physical, chemical and mechanical properties. Accordingly, PBA-a functionalized with polystyrene, poly(methyl methacrylate), polyesters, polyethers, poly(F-caprolactone), polysiloxanes, and polyacetylenes have been synthesized. PBA-a with blended additives including silicates, carbon black, lignin, and nanoparticles and nanotubes are also well-described. For some applications, improved flexural and impact properties in P(BA-a)s would be desirable and could be achieved by the incorporation of complementary low glass transition temperature (T_{g}) polymer. However, curing benzoxazine monomers in a polymer matrix often leads to phase separated polymer-polymer blends due to reduced entropic contributions as the molecular weight of the PBA-a increases. Previously for example, solutions of a benzoxazine monomer in α,ω-dihydroxypolyethyleneglycol (α,ω-HO-PEG-OH) were studied for miscibility and subsequent thermal ROP. In this case, cured solutions afforded a phase separated blend of PBA-a and α,ω-HO-PEG-OH, where the phenolic hydroxyl groups of the PBA-a were where in a non-associated form, i.e., lacking significant hydrogen-bonding within the PBA-a network or to the PEG phase. Less well-described are blends where the complement polymer is capable of several roles such as solubilizing the benzoxazine monomer, accelerating its cure and controllably grafting to the resulting PBA-a network so as to permit adjustment of the chemical and thermal properties of the hybridized material.

Thus, blends that solubilize benzoxazine monomers, accelerate its cure, and can provide controlled structures are needed. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a polybenzoxazine-containing composition, including a polybenzoxazine; and a covalently-grafted polymer onto the polybenzoxazine, a covalently-grafted particle onto the polybenzoxazine, a covalently-grafted fiber onto the polybenzoxazine, or any combination thereof. The covalently-grafted polymer is different from the polybenzoxazine.

In another aspect, the present disclosure features a polymer-grafted polybenzoxazine, including a polybenzoxazine, and a polymer covalently grafted onto the polybenzoxazine, wherein the polymer is different from the polybenzoxazine.

In another aspect, the present disclosure features a method of making a polymer-grafted polybenzoxazine, including providing a blend of a benzoxazine-containing monomer and a polymer functionalized with at least one leaving group; and curing the blend at a predetermined temperature to provide the polymer-grafted polybenzoxazine of the present disclosure.

In yet another aspect, the present disclosure features a polybenzoxazine composite, including a polybenzoxazine, and a particle and/or a fiber covalently grafted onto the polybenzoxazine.

In a further aspect, the present disclosure features a method of making the polybenzoxazine composite, including providing a blend of a benzoxazine-containing monomer and a particle and/or fiber functionalized with at least one leaving group; and curing the blend at a predetermined temperature to provide the polybenzoxazine composite of the present disclosure.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A shows DSC curves from the first heat cycle after quenching (exothermic direction is up) of BA-a blends with mPEGOH₂₀₀₀. Curves have been offset for clarity.

FIG. 1B shows DSC curves from the first heat cycle after quenching (exothermic direction is up) of BA-a blends with mPEGOTs₂₀₀₀. Curves have been offset for clarity.

FIG. 1C shows DSC curves for the percent crystallinity of (c) mPEGOH₂₀₀. The dash line represents the theoretical crystallinity of PEG assuming no interaction with non-crystallizable BA-a.

FIG. 1D shows DSC curves for the percent crystallinity of (d) mPEGOTs₂₀₀₀. The dash line represents the theoretical crystallinity of PEG assuming no interaction with non-crystallizable BA-a.

FIG. 1E shows DSC curves for the percent crystallinity of (e) mPEGOTs₉₀₀. The dash line represents the theoretical crystallinity of PEG assuming no interaction with non-crystallizable BA-a.

FIG. 2A shows DSC curves of the cure study of quenched blends of benzoxazine with mPEGOH₂₀₀₀.

FIG. 2B shows DSC curves of the cure study of quenched blends of benzoxazine with mPEGOTs₂₀₀₀.

FIG. 2C shows DSC curves of the cure study of quenched blends of benzoxazine with mPEGOTs₉₀₀.

FIG. 2D shows a plot for the percent cure of blends of benzoxazine with mPEGOH₂₀₀₀. The dash line represents the theoretical cure of benzoxazine assuming no interaction with PEG.

FIG. 2E shows a plot for the percent cure of blends of benzoxazine with mPEGOTs₂₀₀₀ (●) and mPEGOTs₉₀₀ (▴). The dash line represents the theoretical cure of benzoxazine assuming no interaction with PEG.

FIG. 2F shows a plot for the T_{g} of the PBA-a for cured samples from the mPEGOTs₉₀₀ series. The theoretical curve superimposed over the data points is the result of fitting a Gordon-Taylor relation.

FIG. 3A shows FTIR spectra (C—H and O—H stretch region) for cured blends of BA-a with mPEGOH₂₀₀₀. The minima for the OH stretch signal for selected samples are connected by dotted and dashed lines in each figure. The dotted line at 3372 cm⁻¹ tracks the wavenumber corresponding to the highest signal for the hydroxyl-region and indicates that the phenolic residues in the mPEGOH series are largely free of H-bonding.

FIG. 3B shows FTIR spectra (C—H and O—H stretch region) for cured blends of BA-a with mPEGOTs₂₀₀₀. The minima for the OH stretch signal for selected samples are connected by dotted and dashed lines in each figure. The dashed line at 3245 cm⁻¹ tracks the wavenumber corresponding to the highest signal for the hydroxyl-region and indicates that the phenolic residues in the mPEGOTs series have significant H-bonding.

FIG. 4A shows FTIR tracer plots for S-OH stretch at 885 cm⁻¹ of the effluent from PBA-a blends with mPEGOH₉₀₀.

FIG. 4B shows FTIR tracer plots for S-OH stretch at 885 cm⁻¹ of the effluent from PBA-a blends with mPEGOTs₉₀₀ from thermogravimetric analysis.

FIG. 4C shows the generation of the p-TSA fragment with increasing temperatures as determined with (i) TGA thermogram (ii) Tracer plot of the signal at 885 cm⁻¹ (iii) TGA-MS spectrum scan for m/z 91 and (iv) a DSC thermogram of the 1:1 BA-a:mPEGOTs₉₀₀ blend.

FIG. 5 is a series of SEM images for chloroform extracted PBA-a blends with decreasing mPEGOH₂₀₀₀ (a-g) and mPEGOTs₂₀₀₀ (h-n) content. In (a-g) the original mPEGOH₂₀₀₀ content was 90, 80, 70, 60, 50, 40 and 20 wt %, respectively. In (h-n) the original mPEGOTs₂₀₀₀ content was 90, 80, 70, 60, 50, 40 and 20 wt %, respectively. The scale bar in (h) applies to all SEM images.

FIG. 6A is a graph showing plots for the peak exotherm temperature of cure of BA-a in blends with mPEGOH₂₀₀₀ (▪), mPEGOTs₂₀₀₀ (●), and mPEGOTs₉₀₀ (▴) as a function of PEG wt %. Empty triangles (∇) are used to identify the onset and max cure temperatures of neat BA-a monomer.

FIG. 6B is a graph showing plots for the onset temperature for cure of BA-a in blends with mPEGOH₂₀₀₀ (▪), mPEGOTs₂₀₀₀ (●), and mPEGOTs₉₀₀ (▴) as a function of PEG wt %. Empty triangles (V) are used to identify the onset and max cure temperatures of neat BA-a monomer.

FIG. 7 is graph showing DSC curves of BA-a/mPEGOTs₉₀₀ from the first heat after liquid nitrogen quench. The wt % of PEG in each sample is listed above the data curve. The y-axis is correlated to the curve of the pure mPEGOTs, all other curves have been offset for clarity.

FIG. 8A is an X-ray diffraction pattern of cured BA-a with mPEGOH₂₀₀₀.

FIG. 8B is an X-ray diffraction pattern of cured BA-a with mPEGOH₂₀₀₀, showing the total diffractogram area of FIG. 8A.

FIG. 8C is a crystallite-only diffractogram and its area calculated by fitting and subtracting a background curve for the amorphous halos at ˜19° and 44° in FIG. 8A.

FIG. 8D is a crystallite-only diffractogram and its area calculated by fitting and subtracting a background curve for the amorphous halos at ˜19° and 44° in FIG. 8A.

FIG. 8E is an X-ray diffraction pattern of cured BA-a with mPEGOH₂₀₀₀ each with 40 wt % PEG content.

FIG. 9A is a H-NMR spectra (500 MHz in DMSO-d6) of extractable polymer from cured mPEGOH₂₀₀₀ blends. The original content of PEG are 20, 40, and 50 wt % (i-iii).

FIG. 9B is a H-NMR spectra (500 MHz in DMSO-d6) of extractable polymer from cured mPEGOTs₂₀₀₀ blends. The original content of PEG are 20, 40, and 50 wt % (i-iii).

FIG. 10A is a FTIR tracer plot for sp³ ν_{CH} stretching at 2973 cm⁻¹ from the effluent of PBA-a blends with mPEGOH₉₀₀ from TGA.

FIG. 10B is a FTIR tracer plot for sp³ ν_{CH} stretching at 2973 cm⁻¹ from the effluent of PBA-a blends with mPEGOTs₉₀₀ from TGA.

FIG. 11A is a FTIR tracer plot for ν_{CO} ether stretch at 1136 cm⁻¹ from the effluent of PBA-a blends with mPEGOH₉₀₀ from TGA, and (b) mPEGOTs₉₀₀ from TGA.

FIG. 11B is a FTIR tracer plot for ν_{CO} ether stretch at 1136 cm⁻¹ from the effluent of PBA-a blends with mPEGOTs₉₀₀ from TGA.

FIG. 12A is a FTIR tracer plot for sp² ν_{CH} stretch at 3048 cm⁻¹ from the effluent of PBA-a blends with mPEGOH₉₀₀ from TGA.

FIG. 12B is a FTIR tracer plot for sp² ν_{CH} stretch at 3048 cm⁻¹ from the effluent of PBA-a blends with mPEGOTs₉₀₀ from TGA.

FIG. 13A shows a TGA thermogram for PBA-a blends with mPEGOH₉₀₀.

FIG. 13B shows a TGA thermogram for PBA-a blends with mPEGOTs₉₀₀.

FIG. 13C is a graph showing the T_{D5%} at 475° C. of mPEGOH₉₀₀ blends. Triangles (▴) are used to identify the T_{D5%} and char yield at 475° C. of pure BA-a monomer.

FIG. 13D is a graph showing the T_{D5%} at 475° C. of mPEGOTs₉₀₀ blends. Triangles (▴) are used to identify the T_{D5%} and char yield at 475° C. of pure BA-a monomer.

FIG. 13E is a graph showing the char yield at 475° C. of mPEGOH₉₀₀ blends. Triangles (▴) are used to identify the T_{D5%} and char yield at 475° C. of pure BA-a monomer.

FIG. 13F is a graph showing the char yield at 475° C. of mPEGOTs₉₀₀ blends. Triangles (▴) are used to identify the T_{D5%} and char yield at 475° C. of pure BA-a monomer.

FIG. 14 The generation of the p-TSA fragment with increasing temperature as determined with (i) TGA thermogram, (ii) FTIR tracer plot of the signal at 885 cm⁻¹ from the TGA effluent, (iii) TGA-MS spectrum scan for m/z 91 of the blend sample (●) and pure BA-a (▾), and (iv) a DSC thermogram of the 1:1 BA-a:mPEGOTs₉₀₀ blend.

FIG. 15 High resolution SEM images for chloroform extracted PBA-a blends with decreasing mPEGOH₂₀₀₀ (a-d) and mPEGOTs₂₀₀₀ (e-h) content. In (a-d) the original mPEGOH₂₀₀₀ content was 90, 70, 50, and 20 wt %, respectively. In (e-h) the original mPEGOTs₂₀₀₀ content was 0, 70, 50, and 20 wt %, respectively. For highly porous samples, images were acquired between large void spaces. The scale bar in (a) applies to all SEM images.

FIG. 16 shows the chemical structures of BA-a, HOTs, PSUGH, and PSUOTs.

FIG. 17A shows DSC traces (first cool cycle) for embodiments of a blend of the present disclosure (α,ω-PSU(OH)₂/BA-a).

FIG. 17B shows DSC traces (first cool cycle) for embodiments of a blend of the present disclosure (α,ω-PSU(OTs)₂/BA-a).

FIG. 17C shows plots for glass transition versus the nominal PSU weight content for embodiments of a blend of the present disclosure (α,ω-PSU(OH)₂/BA-a). A curve has been applied by fitting the data to a Gordon-Taylor relation with fitting constant (k) of 0.29±0.03.

FIG. 17D shows plots for glass transition versus the nominal PSU weight content for embodiments of a blend of the present disclosure (α,ω-PSU(OTs)₂/BA-a). A curve has been applied by fitting the data to a Gordon-Taylor relation with fitting constant 0.33±0.03.

FIG. 18A shows DSC traces (first heating cycle) depicting cure exotherm of BA-a in embodiments of blends of the present disclosure (with α,ω-PSU(OH)₂).

FIG. 18B shows DSC traces (first heating cycle) depicting cure exotherm of BA-a in embodiments of blends of the present disclosure (with α,ω-PSU(OTs)₂)

FIG. 18C shows the onset for the cure exotherm of the thermal ROP of BA-a plotted versus the nominal PSU weight content in embodiments of the blends of the present disclosure.

FIG. 18D shows the peak temperature for the cure exotherm of the thermal ROP of BA-a plotted versus the nominal PSU weight content in embodiments of the blends of the present disclosure.

FIG. 18E shows plots for the percent cure of BA-a in blends with α,ω-PSU(OH)₂ (▪) and α,ω-PSU(OTs)₂ (●) as a function of nominal PSU weight content in embodiments of the blends of the present disclosure. The dashed line represents the theoretical cure of BA-a assuming no interaction with PSU.

FIG. 19A shows the glass transition region of DSC traces (second heating cycle) for embodiments of a blend of the present disclosure (α,ω-PSU(OH)₂/PBA-a).

FIG. 19B shows the glass transition region of DSC traces (second heating cycle) for embodiments of a blend of the present disclosure (α,ω-PSU(OTs)₂/PBA-a).

FIG. 19C shows the Gordon-Taylor fits for glass transition for embodiments of a blend of the present disclosure (α,ω-PSU(OH)₂/PBA-a (k=0.01)).

FIG. 19D shows the Gordon-Taylor fits for glass transition for embodiments of a blend of the present disclosure (α,ω-PSU(OTs)₂/PBA-a (k=0.43±0.05)). Open circle data point indicates the theoretical T_{g} for PSU in P(BA-a)-graft-PSU. Crossed-circle data point in indicates the T_{g} for pure PSU in α,ω-PSU(OTs)₂.

FIG. 20 shows SEM images of (a-c) extracted α,ω-PSU(OH)₂/PBA-a cured blends where the nominal α,ω-PSU(OH)₂ content was 80, 50 and 20 wt. %, respectively; (d-f) extracted α,ω-PSU(OTs)₂/PBA-a cured blends where the nominal α,ω-PSU(OTs)₂ content was 80, 50 and 20 wt. %, respectively; and (g) SEM image of extracted PBA-a. The scale bar in (g) applies to all images.

### DETAILED DESCRIPTION

The present disclosure features blends of benzoxazine-containing monomers with polymer having one or more leaving groups, such as tosylated polymers (e.g., tosylated polyethylene glycol (PEG), tosylated polysulfones); or blends of benzoxazine-containing monomers with particles/fibers having one or more leaving groups. The leaving groups of the present disclosure can include, for example, tosylate, perfluoroalkylsufonates (triflates), mesylates, and halides (e.g., iodide, bromide, chloride). Once blended, the benzoxazine-containing monomers can be polymerized (e.g., cured) together with the polymer to form a network or to form a polybenzoxazine grafted with the polymer; or in the case of particles/fibers, the benzoxazine-containing monomers can be polymerized (e.g., cured) together with the particles/fibers to form a bonded composite. As used herein, a polybenzoxazine is used interchangeably with “benzoxazine-containing polymer,” and encompasses a polymer that includes one or more benzoxazine-based or benzoxazine-derived repeating units.

In some embodiments, the present disclosure features a polybenzoxazine-containing composition, including a polybenzoxazine; and a covalently-grafted polymer onto the polybenzoxazine, a covalently-grafted particle onto the polybenzoxazine, a covalently-grafted fiber onto the polybenzoxazine, or any combination thereof. The covalently-grafted polymer is different from the polybenzoxazine.

The blends can be made by melt blending, co-solvating, and/or extrusion. For example, blends can be obtained by melt-blending, which includes weighing the benzoxazine monomer, polymer, and/or particle/fiber and mixing the components, homogenizing the mixture by mortar and pestle or the like, and degassing the mixture in a molten state under reduced pressure (for example by using a low pressure oven). The preparation of blends by co-solvating can occur by providing acetone solutions of benzoxazine monomer and polymer or particle/fiber (having one or more leaving groups), combining appropriate volumes of each solution, and drying the mixture of solutions (via low pressure evaporation of solvent and/or oven dried under reduced pressure; using a rotary evaporator, and/or a vacuum oven). The preparation of blends by extrusion can occur by combining a benzoxazine monomer, a polymer, and/or a particle/fiber in a hopper of an extruder; using a predetermined screw-type, extruding screw rotation rate, and barrel temperature to homogenize the mixture; and capturing the extruded mixture from the extruder nozzle.

The polymers having one or more leaving groups can be linear, and have leaving groups on one or both termini; or can have leaving groups pendant on the polymer backbone, for example pendant on a repeating unit, or on repeat segments (e.g., on segments of a block copolymer); or the leaving groups can be randomly situated on the polymer chain.

The particles/fibers in the blends and/or compositions can have one or more leaving groups on their surfaces or in interstitial spaces.

In some embodiments, the polybenzoxazine and/or polymers having one or more leaving groups can be branched, multi-branched, or dendritic. In some embodiments, the polymer having one or more leaving groups is PEG, polyethers (e.g., polyoxomethylene, (POM)), polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters (e.g., poly(lactic acids) (PLAs), polycaprolactone (PCL), polyterephtalate (PET)), polyurethanes (PU), bimaleimide polymers (BMIs), fluorinated polymers (e.g., polyvinylidenedifluoride (PVDF), polytetrafluoroethylene (PTFE)), vinylics (e.g., polystyrene (PS), polyacrylates (PAcr), polyacrylonitrile (PAN), polyvinylalcohol (PVA), polybutadiene (PB)), conjugated polymers (e.g. polyacetylenes (PAc), polyanilines (PANI), polythiophenes (PTh), poly(pyrrole)s (PPy), polycarbazoles (PCa), polyfluorenes (PFl), poly(p-phenylene vinylene)s (PPV) and/or copolymers thereof. In some embodiments, the polymer having one or more leaving groups is PEG, polysulfones, poly(phenylene oxide), poly(lactic acid), chitosan, cellulosic polymers, fluorinated polymers (e.g., polytetrafluoroethylene (PTFE)) and/or copolymers thereof, each having one or more leaving groups. In some embodiments, the particles/fibers can be further functionalized with a polymer such as PEG, polyethers (e.g., polyoxomethylene, (POM)), polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters (e.g., poly(lactic acids) (PLAs), polycaprolactone (PCL), polyterephtalate (PET)), polyurethanes (PU), bimaleimide polymers (BMIs), fluorinated polymers (e.g., polyvinylidenedifluoride (PVDF), polytetrafluoroethylene (PTFE)), vinylics (e.g., polystyrene (PS), polyacrylates (PAcr), polyacrylonitrile (PAN), polyvinylalcohol (PVA), polybutadiene (PB)), conjugated polymers (e.g. polyacetylenes (PAc), polyanilines (PANI), polythiophenes (PTh), poly(pyrrole)s (PPy), polycarbazoles (PCa), polyfluorenes (PFl), poly(p-phenylene vinylene)s (PPV) and/or copolymers thereof, which in turn can optionally be functionalized with one or more leaving groups. In some embodiments, the polymer is poly(ethylene glycol) and/or polysulfone (PSU), functionalized with one or more leaving groups. In some embodiments, the polymer is poly(ethylene glycol), polysulfone, poly(phenylene oxide), poly(lactic acid), poly(glycolic acid), and/or a fluoropolymer functionalized with one or more leaving groups. In certain embodiments, the polymer does not include poly(ethylene glycol).

In some embodiments, the polymer in the composition is a covalently bound PEG, polyethers (e.g., polyoxomethylene, (POM)), polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters (e.g., poly(lactic acids) (PLAs), polycaprolactone (PCL), polyterephtalate (PET)), polyurethanes (PU), bimaleimide polymers (BMIs), fluorinated polymers (e.g., polyvinylidenedifluoride (PVDF), polytetrafluoroethylene (PTFE)), vinylics (e.g., polystyrene (PS), polyacrylates (PAcr), polyacrylonitrile (PAN), polyvinylalcohol (PVA), polybutadiene (PB)), conjugated polymers (e.g. polyacetylenes (PAc), polyanilines (PANI), polythiophenes (PTh), poly(pyrrole)s (PPy), polycarbazoles (PCa), polyfluorenes (PFl), poly(p-phenylene vinylene)s (PPV) and/or copolymers thereof. In some embodiments, the polymer in the composition is a covalently bound PEG, polysulfones, poly(phenylene oxide), poly(lactic acid), chitosan, cellulosic polymers, fluorinated polymers (e.g., polytetrafluoroethylene (PTFE)) and/or copolymers thereof. In some embodiments, in the composition, the particles/fibers can be further functionalized with a polymer such as PEG, polyethers (e.g., polyoxomethylene, (POM)), polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters (e.g., poly(lactic acids) (PLAs), polycaprolactone (PCL), polyterephtalate (PET)), polyurethanes (PU), bimaleimide polymers (BMIs), fluorinated polymers (e.g., polyvinylidenedifluoride (PVDF), polytetrafluoroethylene (PTFE)), vinylics (e.g., polystyrene (PS), polyacrylates (PAcr), polyacrylonitrile (PAN), polyvinylalcohol (PVA), polybutadiene (PB)), conjugated polymers (e.g. polyacetylenes (PAc), polyanilines (PANI), polythiophenes (PTh), poly(pyrrole)s (PPy), polycarbazoles (PCa), polyfluorenes (PFl), poly(p-phenylene vinylene)s (PPV) and/or copolymers thereof, which in turn can optionally be functionalized with one or more leaving groups. In some embodiments, the polymer in the composition is poly(ethylene glycol) and/or polysulfone (PSU). In some embodiments, the polymer in the composition is poly(ethylene glycol), polysulfone, poly(phenylene oxide), poly(lactic acid), poly(glycolic acid), and/or a fluoropolymer. In certain embodiments, the polymer does not include poly(ethylene glycol).

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “aryl” refers to monocyclic or polycyclic (e.g., having 2, 3, or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 30, from 2 to about 24, from 2 to about 20, from 2 to about 10, from 2 to about 8, from 2 to about 6, or from 2 to about 4 carbon atoms.

As used herein, “alkynylene” refers to a linking alkynyl group.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “haloalkenyl” refers to an alkenyl group having one or more halogen substituents.

As used herein, “haloalkynyl” refers to an alkynyl group having one or more halogen substituents.

As used herein, “haloalkoxy” refers to an —O-(haloalkyl) group.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “arylene” refers to a linking aryl group.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent.

Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like.

As used herein, “cycloalkylene” refers to a linking cycloalkyl group.

As used herein, “heteroalkyl” refers to an alkyl group having at least one heteroatom such as sulfur, oxygen, or nitrogen.

As used herein, “heteroalkylene” refers to a linking heteroalkyl group.

As used herein, a “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heteroarylene” refers to a linking heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer while the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” or “repeating unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “neutral net charge” is defined as a polymer having a net charge that is less than 50 percent (e.g., less than 20 percent, less than 10 percent, less than 5 percent, or less than 2 percent) of the anionic or cationic groups content on the polymer chain. In some embodiments, a neutral net charge polymer is uncharged.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, “living polymerization” refers to a method of synthesizing polymers using the well-known concept of addition polymerization, that is, polymerization wherein monomers are added one-by-one to an active site on the growing polymer chain but one wherein the active sites for continuing addition of another monomer are never fully eliminated other than on purpose. That is, the polymer chain is virtually always capable of further extension by the addition of more monomer to the reaction mixture unless the polymer has been capped, which can be reversible so as permit polymerization to continue or quenched, which is usually permanent. While numerous genera of living polymerizations are known, currently the predominant types are anionic, cationic, and radical living polymerizations. Radical polymerization involves a free radical initiator that extracts one of the pi electrons of the double bond of an ethylenic monomer resulting in a reactive unpaired electron on the carbon at the other end of the former double bond from that with which the initiator reacted. The unpaired electron then reacts with the double bond of another monomer creating a stable sigma bond and another free radical and so on. With conventional initiators the sequence is eventually stopped by a termination reaction, generally a combination reaction in which the unpaired electrons of two propagating chains combine to form a stable sigma bond or a disproportionation in which a radical on an active chain strips a hydrogen atom from another active chain or from an impurity in the reaction mixture to produce a stable unreactive molecule and a molecule containing a double bond. In a living polymerization, the ability of the growing chains to enter into a termination reaction is eliminated, effectively limiting the polymerization solely by the amount of monomer present; that is, the polymerization continues until the supply of monomer has been exhausted. At this point the remaining free radical species become substantially less active due to capping of the free radical end group with such entities as, without limitation, nitroxyl radicals, halogen molecules, oxygen species such as peroxide and metals or simply by interaction with solvent and the like. If, however, more monomer is added to the solution, the polymerization reaction can resume except as noted above.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Benzoxazine-Containing Blends, Polymer Networks Thereof, and Composites Thereof

As used herein, a benzoxazine-containing monomer or polymer describes any monomer or polymer that contains, or that is derived from, one or more 1,3-benzoxazine with substituents on the oxazine or benzene ring. For example, the benzoxazine-containing monomer or polymer can contain a single 1,3-benzoxazine moiety, two 1,3-benzoxazines moieties, three 1,3-benzoxazines moieties, or more. Examples of suitable benzoxazine moieties are shown below, where R¹ to R⁷ represent different substituents on the monomer:

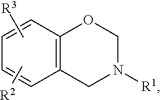

a representative monomer containing a single 1,3-benzoxazine;

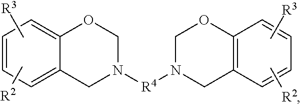

a representative monomer containing two 1,3-benzoxazines;

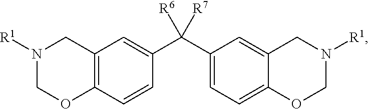

a representative monomer containing two 1,3-benzoxazines bridged through the benzyl unit; and

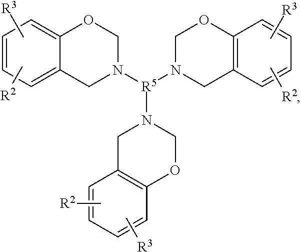

a representative monomer containing three 1,3-benzoxazines,

wherein

R¹, R², R³ are each independently selected from halo, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₁₋₆ alkoxy, C₁₋₆ haloalkoxy, aryl, heteroaryl, cycloalkyl, heterocycloalkyl, CN, NO₂, and OH;

R⁴ is selected from alkylene, alkenylene, arylene, haloalkenylene, cycloalkylene, heteroalkylene, and heteroarylene;

R⁵ is selected from alkylene, alkenylene, arylene, haloalkenylene, cycloalkylene, heteroalkylene, and heteroarylene; and

R⁶ and R⁷ are each independently selected from hydrogen, alkyl, C₁₋₆ haloalkyl, aryl, heteroaryl, cycloalkyl, heterocycloalkyl.

In some embodiments, R¹, R², R³ are each independently selected from halo, C₁₋₆ alkyl, C₁₋₆ haloalkyl, C₁₋₆ alkoxy, C₁₋₆ haloalkoxy, aryl, heteroaryl, cycloalkyl, and heterocycloalkyl.

In some embodiments, R¹, R², R³ are each independently selected from C₁₋₆ alkyl, C₁₋₆ haloalkyl, C₁₋₆ alkoxy, C₁₋₆ haloalkoxy, aryl, heteroaryl, cycloalkyl, and heterocycloalkyl.

In some embodiments, R¹, R², R³ are each independently selected from C₁₋₆ alkyl, C₁₋₆ alkoxy, aryl, and cycloalkyl.

In some embodiments, R¹, R², R³ are each independently selected from C₁₋₆ alkyl and aryl.

In some embodiments, R¹, R², R³ are each independently selected from C₁₋₆ alkyl.

In some embodiments, R¹, R², R³ are each independently selected from methyl and ethyl.

In some embodiments, R¹, R², R³ are each independently methyl.

In some embodiments, R⁴ is selected from alkenylene, arylene, haloalkenylene, cycloalkylene, heteroalkylene, and heteroarylene.

In some embodiments, R⁴ is selected from alkenylene, arylene, and heteroarylene.

In some embodiments, R⁴ is selected from alkenylene and arylene.

In some embodiments, R⁴ is alkenylene.

In some embodiments, R⁵ is selected from alkenylene, arylene, haloalkenylene, cycloalkylene, heteroalkylene, and heteroarylene.

In some embodiments, R⁵ is selected from alkenylene, arylene, and heteroarylene.

In some embodiments, R⁵ is selected from alkenylene and arylene.

In some embodiments, R⁵ is alkenylene.

In some embodiments, R⁶ and R⁷ are each independently selected from hydrogen, alkyl, C₁₋₆ haloalkyl, aryl, and heteroaryl.

In some embodiments, R⁶ and R⁷ are each independently selected from hydrogen, alkyl, C₁₋₆ haloalkyl, and aryl.

In some embodiments, R⁶ and R⁷ are each independently selected from hydrogen, alkyl, and aryl.

In some embodiments, R⁶ and R⁷ are each independently selected from hydrogen and alkyl.

In some embodiments, R⁶ and R⁷ are each hydrogen.

In some embodiments, the benzoxazine-containing monomer of the present disclosure can have one or more benzoxazine moieties covalently attached to the termini of a polymer, or pendant on a repeating unit of a polymer. The polymer can be, for example, POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, and PPV.

The benzoxazine-containing monomer is blended, then cured (e.g., polymerized), with a polymer having one or more leaving groups, and/or with a particle/fiber having one or more leaving groups, as described above.

Thus, in some embodiments, the benzoxazine-containing monomer of the present disclosure is:

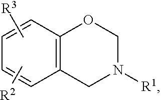

a representative monomer containing a single 1,3-benzoxazine;

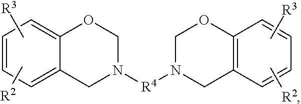

a representative monomer containing two 1,3-benzoxazines;

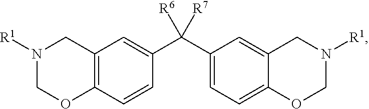

a representative monomer containing two 1,3-benzoxazines bridged through the benzyl unit; and

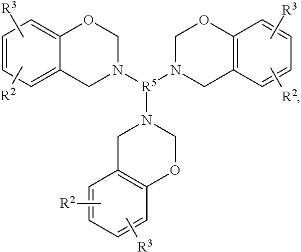

a representative monomer containing three 1,3-benzoxazines,

wherein, R¹, R², R³ are each independently selected from halo, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₁₋₆ alkoxy, C₁₋₆ haloalkoxy, aryl, heteroaryl, cycloalkyl, heterocycloalkyl, CN, NO₂, OH, and a polymer and oligomer (such as POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, and PPV);

R⁴ is selected from alkylene, arylene, and linking polymer and oligomer (e.g., linking POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, and PPV bound at two locations to the benzoxazol units), and

R⁵ is selected from alkylene, arylene, and linking polymer and oligomer (e.g., linking POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, PPV bound at two locations to the benzoxazole units);

R⁶ and R⁷ are each independently selected from hydrogen, alkyl, aryl, and a polymer and oligomer (such as POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, and PPV).

In some embodiments, the benzoxazine monomer is 4-dihydro-2H-1,3-benzoxazine, 3-phenyl-3,4-dihydro-2H-1,3-benzoxazine, 3,4-dihydro-3-methyl-2H-1,3-benzoxazine, 3,4-dihydro-3,8-dimethyl-2H-1,3-benzoxazine, 3-(4-methylphenyl)-3,4-dihydro-2H-1,3-benzoxazine, 3-(4-methylolphenyl)-3,4-dihydro-2H-1,3-benzoxazine, 6-methyl-3,4-dihydro-3-phenyl-2H-1,3-benzoxazine, 6-chloro-3,4-dihydro-3-phenyl-2H-1,3-benzoxazine, 6-(methylol)-3,4-dihydro-3-phenyl-2H-1,3-benzoxazine, 6,6′-(1-methylethylidene)bis[3,4-dihydro-3-phenyl-2H-1,3-benzoxazine (bisphenol-A based benzoxazine), 6,6′-methylenebis[3,4-dihydro-3-phenyl-2H-1,3-benzoxazine (bisphenol-F based benzoxazine), 3-(2-furanylmethyl)-3,4-dihydro-2H-1,3-benzoxazine, and/or 6,6′-(1-methylethylidene)bis[3-(2-furanylmethyl)-3,4-dihydro-2H-1,3-benzoxazine.

The present disclosure also features blends of benzoxazine-containing monomers with polymers that are functionalized with one or more leaving groups, such as one or more as tosylate, perfluoroalkylsufonates (triflates), mesylates, and/or halides (e.g., iodide, bromide, chloride). In some embodiments, the leaving groups are selected from tosylate, triflates, or mesylates. In some embodiments, the leaving groups are tosylates. Once blended, the benzoxazine-containing monomers can be polymerized (cured) together with the polymers to form a grafted polymer, where a polybenzoxazine is covalently bonded to the polymers.

Thus, in some embodiments, the present disclosure features a polymer-grafted polybenzoxazine, including a polybenzoxazine, and a polymer covalently grafted onto the polybenzoxazine, wherein the polymer is different from the polybenzoxazine. The polymer can be, for example, poly(ethylene glycol), polyethers (e.g., polyoxomethylene, (POM)), polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters (e.g., poly(lactic acids) (PLAs), polycaprolactone (PCL), polyterephtalate (PET)), polyurethanes (PU), bimaleimide polymers (BMIs), fluorinated polymers (e.g., polyvinylidenedifluoride (PVDF), polytetrafluoroethylene (PTFE)), vinylics (e.g., polystyrene (PS), polyacrylates (PAcr), polyacrylonitrile (PAN), polyvinylalcohol (PVA), polybutadiene (PB)), conjugated polymers (e.g. polyacetylenes (PAc), polyanilines (PANI), polythiophenes (PTh), poly(pyrrole)s (PPy), polycarbazoles (PCa), polyfluorenes (PFl), poly(p-phenylene vinylene)s (PPV), and/or copolymers thereof. In some embodiments, the polymer is poly(ethylene glycol) (PEG) and/or PSU. In some embodiments, the polymer is poly(ethylene glycol), polysulfone, poly(phenylene oxide), poly(lactic acid), poly(glycolic acid), and/or a fluoropolymer. In some embodiments, the polymer is not poly(ethylene glycol).

As described above, the polybenzoxazine polymer can be linear having two termini. The polymer can be covalently grafted onto a terminus of the polybenzoxazine polymer. In some embodiments, the polymer is grafted onto both termini of a linear polybenzoxazine polymer. In some embodiments, the polymer is grafted onto, or further grafted onto, at least one repeating unit of the polybenzoxazine polymer. Thus, the linear polybenzoxazine polymer can be crosslinked by the polymer to provide a network, and/or the polymer-grafted polybenzoxazine can include a brush copolymer. In some embodiments, the polybenzoxazine polymer is branched. The branched polybenzoxazine polymer is crosslinked by the polymer to provide a network.

Before incorporation into a blend with a polybenzoxazine, the polymer can be functionalized with leaving groups. A schematic representation of polymers having positions functionalizable with leaving groups is shown below. In structure (A), both termini X and Y can be functionalized with leaving groups. In some embodiments, the polymer can be terminated only at one end with a leaving group. In structure (B), the polymer is functionalized at positions Z along the backbone with either each repeat unit, at patterned segments, or at random repeating units.

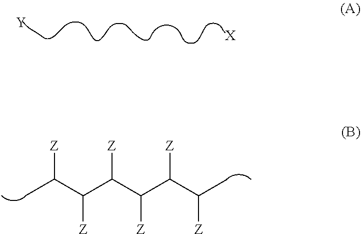

The polymer having positions functionalizable with leaving groups include, for example, POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, and PPV. The POM, PSU, PES, PAES, PPO, PPE, PA, PC, PLA, PCL, PET, PU, BMI, PVDF, PTFE, PS, PAcr, PAN, PVA, PB, PAc, PANI, PTh, PPy, PCa, PFl, and PPV can include one or more leaving groups, such as tosylate, perfluoroalkylsufonates (triflates), mesylates, and/or halides (e.g., iodide, bromide, chloride). In some embodiments, the leaving groups are selected from tosylate, triflates, or mesylates. In some embodiments, the leaving groups are tosylates. In certain embodiments, the leaving group is not tosylate. The leaving groups can functionalize one or more termini of the polymer.

The polymer can be functionalized with a leaving group, for example, as follows. The polymer can be dissolved in a suitable solvent, and reacted with halo derivative of the leaving group in the presence of, for example, a base. For example, to a round bottom flask with a gas inlet was stirred a polymer (e.g., polysulfone) and a solvent (e.g., chloroform) until the polymer is dissolved. An excess of a halo derivative of the leaving group, such as p-toluenesulfonyl chloride, can be added to the solution and dissolved. To the stirred polymer solution a base, such as triethylamine can be added under inert gas. The reaction can be stirred under inert gas at a suitable temperature (e.g., room temperature). The leaving group-functionalized polymer product can then be precipitated, dried, and optionally purified (e.g., by silica chromatography). As another example, mesylation of polyethyleneglycol can proceed by providing a dichloromethane solution of hydroxy-terminated PEG, adding an excess of methanesulfonyl chloride, and a base such as triethylamine. The reaction can proceed until completion. The mesylated PEG can be precipitated in a poor solvent, collected by filtration, and dried under vacuum.

When the polymer functionalized with at least one leaving group is blended with a benzoxazine-containing monomer or polymer, the blend can be cured at a predetermined temperature to provide a composition where the polymer is covalently bound to polybenzoxazine. As described above, the benzoxazine-containing monomer or polymer can include an optionally substituted 1,3-benzoxazine unit. The benzoxazine-containing monomer or polymer can include one, two, or three optionally substituted 1,3-benzoxazine unit.

The present disclosure also features blends of a benzoxazine-containing monomer or polymer with small particles and/or fibers having surfaces functionalized with one or more leaving groups, such as one or more of tosylate, perfluoroalkylsufonates (triflates), mesylates, and/or halides (e.g., iodide, bromide, chloride). In some embodiments, the leaving groups are selected from tosylate, triflates, or mesylates. In some embodiments, the leaving groups are tosylates. Once blended, the benzoxazine-containing monomer or polymer can be polymerized (cured) together with the particles and/or fibers to form a composite, where a polybenzoxazine is covalently bonded to the particles and/or fibers to form a network.

The particles can be 2 nm or more and/or 2 mm or less in maximum dimension, and/or can be spherical, cubic, tetrahedral, rod-like, plate-like, star-like (multi-point), or amorphous in shape. The particles can include, for example, talc, silica, silicates, zeolites, and/or titania particles. As used here, “maximum dimension” refers to the maximum cross-section of a given particle of irregular or regular shape.

The fibers can include those with dimensions of 1 nm-2 mm in width and lengths of greater than 1 nm (e.g., 10-300 mm). In some embodiments, the fibers have a length of 0.01 mm to 1000 mm. The ratio of the width to length can be 1 nm:1 nm or more and/or 1 nm 1000 mm or less.

The particle and/or fiber can further be functionalized with a polymer independently selected from poly(ethylene glycol), polyethers (e.g., polyoxomethylene, (POM)), polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters (e.g., poly(lactic acids) (PLAs), polycaprolactone (PCL), polyterephtalate (PET)), polyurethanes (PU), bimaleimide polymers (BMIs), fluorinated polymers (e.g., polyvinylidenedifluoride (PVDF), polytetrafluoroethylene (PTFE)), vinylics (e.g., polystyrene (PS), polyacrylates (PAcr), polyacrylonitrile (PAN), polyvinylalcohol (PVA), polybutadiene (PB)), conjugated polymers (e.g. polyacetylenes (PAc), polyanilines (PANI), polythiophenes (PTh), poly(pyrrole)s (PPy), polycarbazoles (PCa), polyfluorenes (PFl), poly(p-phenylene vinylene)s (PPV), and/or copolymers thereof. In some embodiments, the polymer is poly(ethylene glycol) (PEG) and/or PSU. In some embodiments, the polymer is poly(ethylene glycol), polysulfone, poly(phenylene oxide), poly(lactic acid), poly(glycolic acid), and/or a fluoropolymer. In some embodiments, the polymer is not poly(ethylene glycol).

A particle and/or fiber can be functionalized by providing a suspension of a reactive group-containing particle and/or fiber (e.g., a hydroxyl-functionalized silica) in a solvent, adding a reactive leaving group that contains, for example, a halo moiety (e.g., methanesulfonyl chloride), an optional catalyst, and reacting the mixture for a period of time to provide the leaving group-functionalized particle and/or fiber. The leaving group-functionalized particle and/or fiber can be purified and isolated by washing with a solvent to remove excess reagents and/or side products, filtering, and drying. For example, to a chilled (0° C.) dry dichloromethane suspension of propoxypropane-1,2-diol-functionalized silica can be added methanesulfonyl chloride and dimethylaminopyridine (DMAP). The reaction can proceed for 96 h before decanting the solvent away from the silica. Rinsing with repeated exposure to fresh acetone and/or DCM and decanting can purify the mesylated silica from the reagents used in the synthesis and any reaction byproducts. The mesylated silica is then collected by filtration and dried under vacuum.

As described above, the benzoxazine-containing polymer can be linear having two termini. The particle and/or fiber can be covalently grafted onto a terminus of the benzoxazine-containing polymer. In some embodiments, the particle and/or fiber is grafted onto both termini of a linear benzoxazine-containing polymer. In some embodiments, the particle and/or fiber is grafted onto, or further grafted onto, at least one repeating unit of the benzoxazine-containing polymer. Thus, the linear benzoxazine-containing polymer can be crosslinked by the particle and/or fiber to provide a network, and/or the particle and/or fiber-grafted benzoxazine-containing can include a brush morphology. In some embodiments, the benzoxazine-containing polymer is branched. The branched benzoxazine-containing polymer is crosslinked by the particle and/or fiber to provide a network.

In some embodiments, blends of the present disclosure can provide thermoset polymer networks or composites, or thermoplastic polymer networks or composites. For example, where benzoxazine-containing monomers or polymers containing two or more benzoxazines is used in a blend, the resulting polymer network or composite can be a thermoset. As another example, where a monomer or polymer containing a single benzoxazine is used in a blend, the resulting polymer network or composite can be a thermoplastic.

In any of the above-mentioned embodiments of blend of a benzoxazine-containing monomer or polymer with polymers, fibers, and/or particles, the blend can further include a polymerization catalyst. Examples of polymerization catalysts can include Lewis acids, such as PCl₅ and AlCl₃. Examples of catalysts can also include organic acids, such as a carboxylic acid-, amine-, imidazole-, or catechol-containing molecule (e.g., 1,6-hexanedioic acid, 1,10-decanedioic acid, 2,2′-dihydroxybiphenyl, 3-aminoaniline, p-toluenesulfonic acid, sodium p-toluenesulfonate, lithium p-toluenesulfonate, methyltosylate cyclohexyltosylate, and/or neopentyl 4-methylbenzenesulfonate). In some embodiments, the blends of the present disclosure can generate one or more polymerization catalysts in situ, during curing (i.e., polymerization). For example, para-toluene sulfonate can be liberated during heating and can serve as a polymerization catalyst for the polymerization of benzoxazine monomers.

In any of the above-mentioned embodiments of blend of a benzoxazine-containing monomer or polymer with polymers, fibers, and/or particles, polymerization of the benzoxazine unit can proceed via ring-opening polymerization. The conditions of the polymerization can influence the molecular weight (M_{w}) of the resulting composition. In the case of a monofunctional benzoxazine monomer or polymer, the polymer molecular weight can range from 1,000-5,000,000 g/mol. In the case of a multi-functional benzoxazine monomer or polymer having 2 or more benzoxazine units, the polymer molecular weight can range from 1,000-infinite g/mol.

In any of the above-mentioned embodiments of blend of benzoxazine-containing monomer or polymer with polymers, fibers, and/or particles having one or more leaving groups, the blends of the present disclosure can omit a solvent. In certain embodiments, the blends of the present disclosure consists essentially of, or consists of a benzoxazine-containing monomer or polymer and a polymer, particle, or fiber having one or more leaving groups, optionally with one or more polymerization catalysts. In certain embodiments, the blends of the present disclosure consists essentially of, or consists of a benzoxazine-containing monomer or polymer with polymers, particles, and/or fibers having one or more leaving groups, optionally with one or more polymerization catalysts. The leaving group-functionalized polymer, particle, or fiber can serve as a solvent for the benzoxazine-containing monomer or polymer, an accelerant for the polymerization of benzoxazine-containing monomer or polymer (e.g., thermal ROP), and/or as a reactant for grafting benzoxazine-containing monomer or polymer. Without wishing to be bound by theory, it is believed that the reaction and covalent bonding between the leaving group-functionalized polymer, particle, or fiber and the benzoxazine-containing monomer or polymer allow for greater tuning of the resulting properties of the grafted polymers or composites based on tuned molecular weight and structure in the leaving group-functionalized polymer.

In some embodiments, the blends of the present disclosure are homogeneous. In some embodiments, the blends can include 0.1 mole % or more (e.g., 0.5 mole % or more, 5 mole % or more, 25 mole % or more, 50 mole % or more, 75 mole % or more, or 90 mole % or more) and/or 99.9 mole % or less (e.g., 90 mole % or less, 75 mole % or less, 50 mole % or less, 25 mole % or less, 5 mole % or less, 0.5 mole % or less, 0.1 mole % or less, or 0.05 mole % or less) of benzoxazine-containing monomer or polymer. In some embodiments, the blends can include 0.01 mole % or more (e.g., 0.05 mole % or more, 0.1 mole % or more, 0.5 mole % or more, 5 mole % or more, 25 mole % or more, 50 mole % or more, or 75 mole % or more) and/or 90 mole % or less (e.g., 75 mole % or less, 50 mole % or less, 25 mole % or less, 5 mole % or less, 0.5 mole % or less, 0.1 mole % or less, or 0.05 mole % or less) of a polymer having one or more leaving groups, relative to the benzoxazine units. In some embodiments, the blends can include 0.1 mole % or more (e.g., 0.5 mole % or more, 5 mole % or more, 25 mole % or more, 50 mole % or more, 75 mole % or more, or 90 mole % or more) and/or 99.9 mole % or less (e.g., 90 mole % or less, 75 mole % or less, 50 mole % or less, 25 mole % or less, 5 mole % or less, 0.5 mole % or less, 0.1 mole % or less, or 0.05 mole % or less) of a particle and/or fiber having one or more leaving groups, relative to the benzoxazine units. The total percentages of the components of the blend add to 100 mole %.

In some embodiments, the blends of the present disclosure described above can be tuned to reduce the curing temperature (i.e., the temperature at which the benzoxazine polymerizes), with an increase in the enthalpy of cure due to higher monomer conversion and/or the grafting polymers or particles onto the benzoxazine-containing network.

In some embodiments, the blends of the present disclosure can be heated at a temperature of 25° C. or more (e.g., 50° C. or more, 100° C. or more, 200° C. or more, 300° C. or more, or 400° C. or more) and/or 500° C. or less (e.g., 400° C. or less, 300° C. or less, 200° C. or less, 100° C. or less, or 50° C. or less). In some embodiments, the blends of the present disclosure can be heated for a duration of 1 minute or more (e.g., 10 minutes or more, 30 minutes or more, 1 hour or more, 6 hours or more, 12 hours or more, 24 hours or more, or 36 hours or more) and/or 48 hours or less (e.g., 36 hours or less, 24 hours or less, 12 hours or less, 6 hours or less, 1 hour or less, 30 minutes or less, or 10 minutes or less).

The cured blends can be porous and have a void volume of 0.01% to 90% by volume (e.g., 5% to 80% by volume, 5% to 60% by volume, 10% to 80% by volume, 10% to 60% by volume, 10% to 40% by volume, 10% to 30% by volume, or 20% to 30% by volume). In some embodiments, the cured blends have a void volume of 0.01% or more (e.g., 0.5% or more, 5% or more, 25% or more, 50% or more, or 75% or more) and/or 90% or less (e.g., 75% or less, 50% or less, 25% or less, 5% or less, 0.5% or less, or 0.1% or less) by volume. In some embodiments, the void volume can be tuned by varying the blend composition. The ability to tune the void content can be used for tuning transparency, macroscopic density, and strength characteristics in cured macroscopic parts made from the blends.

The blends, and resulting polymer networks and composites of the present disclosure can be used in a variety of applications, such as in biomedical plastics; electronics; industrial and/or aerospace fiberglass; carbon fiber composites membranes; and/or membranes for water purification, gas purification, ion purification, and batteries (e.g., for conducting Li ions).

### Synthesis

The compounds of the present disclosure can be prepared in a variety of ways known to one skilled in the art of organic synthesis. The compounds of the present disclosure can be synthesized using the methods as hereinafter described below, together with synthetic methods known in the art of synthetic organic chemistry or variations thereon as appreciated by those skilled in the art.

The compounds of this disclosure can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art, for example, chromatography (medium pressure) on a suitable adsorbent (e.g., silica gel, alumina and the like) HPLC, or preparative thin layer chromatography; distillation; sublimation, trituration, or recrystallization.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene's Protective Groups in Organic Synthesis, 4^{th}Ed., John Wiley & Sons: New York, 2006, which is incorporated herein by reference in its entirety.

The reactions of the processes described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the reaction step, suitable solvent(s) for that particular reaction step can be selected. Appropriate solvents include water, alkanes (such as pentanes, hexanes, heptanes, cyclohexane, etc., or a mixture thereof), aromatic solvents (such as benzene, toluene, xylene, etc.), alcohols (such as methanol, ethanol, isopropanol, etc.), ethers (such as dialkylethers, methyl tert-butyl ether (MTBE), tetrahydrofuran (THF), dioxane, etc.), esters (such as ethyl acetate, butyl acetate, etc.), halogenated solvents (such as dichloromethane (DCM), chloroform, dichloroethane, tetrachloroethane), dimethylformamide (DMF), dimethylsulfoxide (DMSO), acetone, acetonitrile (ACN), hexamethylphosphoramide (HMPA) and N-methylpyrrolidone (NMP). Such solvents can be used in either their wet or anhydrous forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

The compounds of the disclosure can be prepared, for example, using the reaction pathways and techniques as described below and in the Examples.

For example, as a representative synthesis of benzoxazine monomer, the synthesis of (3-phenyl-3,4-dihydro-2H-1,3-benzoxazin-6-yl)methanol (pHBA-a) is presented. To a 100 mL round bottom flask with a gas inlet aniline (2.00 mL, 21.5 mmol), p-hydroxybenzyl alcohol (2.50 g, 20.1 mmol), and paraformaldehyde (1.21 g, 40.3 mmol) are added. The solids are dissolved in toluene (30 mL) and stirred under nitrogen at 100° C. for 8 hours. The reaction mixture is then concentrated in vacuo after the solids were removed and dissolved in chloroform (30 mL). The solution is then washed with NaHCO₃ (0.5 M, 3×30 mL) and deionized water (30 mL) before drying over Na₂SO₄. The product is allowed to crystallize in the chloroform solution at 0° C. and collected as a white solid. The product can be characterized by proton nuclear magnetic resonance. Substituted benzoxazine monomers can be readily made by one skilled in the art.

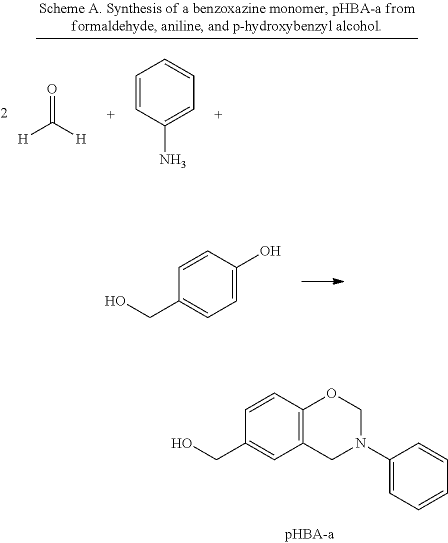 [SMILES: C=O ; Nc1ccccc1 ; OCc1ccc(O)cc1 ; OCc1ccc2c(c1)CN(c1ccccc1)CO2]

General procedures for the synthesis of a polymer functionalized with leaving groups are described above. A representative synthesis of a polymer functionalized with leaving groups is provided as follows. To a round bottom flask with a gas inlet a methoxy-PEGOH (mPEGOH) polymer can be dissolved in a solvent, such as dichloromethane. To the flask a base, such as pyridine and an excess of p-toluenesulfonyl chloride are added. The reaction is stirred under nitrogen at room temperature until reaction completion. The reaction mixture is concentrated in vacuo and dissolved in a solvent such as diethyl ether before being chromatographed on a column packed with neutralized alumina. The fractions can be collected, and concentrated in vacuo. The tosylated product (mPEGOTs) can be characterized by proton nuclear magnetic resonance (H NMR).

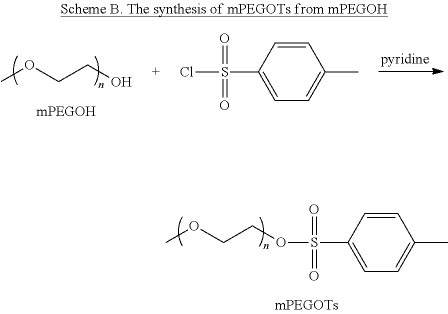

General procedures for the synthesis of a particle/fiber functionalized with leaving groups are described above. A representative synthesis of a particle functionalized with leaving groups is provided as follows. To a chilled (0° C.) dry DCM suspension of propoxypropane-1,2-diol-functionalized silica is added methanesulfonyl chloride. A DCM solution of a catalyst, such as dimethylaminopyridine (DMAP), is then added slowly to this rapidly stirred solution. The reaction proceeds for 96 h before decanting the solvent away from the silica. Rinsing with solvent, such as acetone and/or DCM, and decanting purifies the mesylated silica from the reagents used in the synthesis and any reaction byproducts. The mesylated silica is collected by filtration and dried under vacuum.

A general procedure for forming and curing blends is as follows. Blends can be deposited into molds, such as silicone molds (for example with dimensions of 60×6×1 mm) and then heated at lower temperatures (e.g., 50-125° C. for about 4 h) in a vacuum oven to remove any residual solvents. The temperature can then be elevated (e.g., to about 225° C.) and the bends allowed to cure (e.g., for about 2-3 h) while still under vacuum. A representative synthesis of a silica blend is as follows: silica blends can be dissolved in minimal amounts of dichloromethane and stirred to maintain good dispersion of the particles before being solvent casted into the similar molds. The silica blends can then be heated at 50-150° C. for 20 min to remove the majority of the solvent, and then allowed to heat at 150° C. for an additional 3-6 h under vacuum to remove residual solvent. The silica blends can then be cured at 225° C. for 2-3 h under vacuum. A blend including fibers can be synthesized in a similar manner.

In some embodiments, curing the blend can occur at a temperature of 150° C. or more (e.g., 200° C. or more, 225° C. or more, 250° C. or more, 275° C. or more, 300° C. or more, 325° C. or more, 350° C. or more, or 375° C. or more) and/or 400° C. or less (e.g., 375° C. or less, 350° C. or less, 325° C. or less, 300° C. or less, 275° C. or less, 250° C. or less, 225° C. or less, or 200° C. or less). Curing can occur for a duration of 30 minutes or more (e.g., 1 hour or more, 2 hours or more, 3 hours or more, or 4 hours or more) and/or 5 hours or less (e.g., 4 hours or less, 3 hours or less, 2 hours or less, or 1 hour or less).

The blends can be characterized by, for example, differential scanning calorimetry (DSC), x-ray diffraction (XRD), and/or polarized optical microscopy. The cure of the blends can be analyzed by, for example, DSC, UV-vis spectroscopy, thermogravimetric analysis coupled to Fourier transform infrared spectroscopy (TGA-FTIR), and/or thermogravimetric analysis/mass spectrometry (TGA-MS). The as-recovered cured blends can be analyzed by, for example, TGA, scanning electron microscopy (SEM), FTIR, XRD, polarized optical microscopy, gel content, NMR, and/or mechanical tensile tests. Extracts and insoluble mass from the cured blends can be studied by TGA, SEM, FTIR, XRD, polarized optical microscopy, gel content, NMR, and/or mechanical tensile tests.

Example 1 below describes the solubility, polymerization and the macromolecular structure of cured blends of BPA-based benzoxazine (BA-a) in end-group tosylated polyethyleneglycol (mPEGOTs); and their comparisons to analogues from hydroxyl-terminated polyethyleneglycol. As shown in Example 1, BA-a can be homogeneously dispersed in both polymers where a wide loading range of ˜100-40 wt. % BA-a is possible. The cure temperature for blended BA-a/mPEGOTs was up to 50° C. less than that of pure BA-a or mPEGOH blends. Without wishing to be bound by theory, it is believed that nucleophilic attack of BA-a on the end-group of mPEGOTs produces free tosylate and cationic BA-a-based initiators that act as cure catalysts—the former can be detected by TGA-FTIR and TGA-MS. H-NMR and FTIR revealed the polybenzoxazine (PBA-a) synthesized in mPEGOTs include a phenolic rich molecular structure with H-bonded hydroxyl residues and grafted PEG-chains. SEM confirmed that this material, P(BA-a)-graft-mPEGOTs, has a homogeneous microstructure. The glass transition and the thermal stability of the P(BA-a)-graft-mPEGOTs can be tuned based on the blend composition. Example 2 describes blends of bisphenol-a based benzoxazine and end-group tosylated polymers in varying ratios, which were then cured. Cure acceleration, glass transition, and thermal stability of the blends were investigated using differential scanning calorimetry and thermogravimetric analysis. Miscibility and bonding were studied by infrared spectroscopy and NMR. The cured blends were imaged by scanning electron microscopy to determine homogeneity and phase separation.

### EXAMPLES

### Example 1. Pegylated Polybenzoxazine Networks with Increased Thermal Stability from Miscible Blends of Tosylated Poly(Ethylene Glycol) and a Benzoxazine Monomer

Presented herein is a study on the solubility, polymerization and the molecular characterization of blends of BA-a in end-group mono-tosylated polyethyleneglycol (mPEGOTs). More specifically, blends of BPA-based benzoxazine (BA-a) with either α-hydroxyl-terminated (mPEGOH) or tosylate-terminated mPEGOTs were prepared and characterized. Contrary to the mPEGOH blends, a decrease in the cure temperature was found for the BA-a/mPEGOTs combination. Infrared spectroscopy and mass spectroscopy were used to monitor heated blends and have identified free tosylate as a likely catalyst in curing blends of BA-a/mPEGOTs. In contrast to cured mPEGOH blends, H-NMR and FTIR revealed that the PBA-a network synthesized in mPEGOTs had a phenolic rich molecular structure with strongly H-bonded hydroxyl residues and grafted PEG-chains. Scanning electron microscopy confirmed that P(BA-a)-graft-mPEGOTs [P(BA-a)-g-mPEGOTs] was homogeneous in its microstructure. The tuning of the T_{g} and the thermal stability of the P(BA-a)-g-mPEGOTs was also demonstrated in mPEGOTs blend series. This work demonstrated access to well-defined PBA-a alloys from BP-a/polymer blends. Without wishing to be bound by theory, it is believed that the reaction and covalent bonding between curing BA-a and mPEGOTs can apply to other miscible benzoxazine/polymer blends and can allow for greater tuning of the resulting properties of the graft copolymers based on tuned molecular weight and structure in the initial tosylated-polymer.

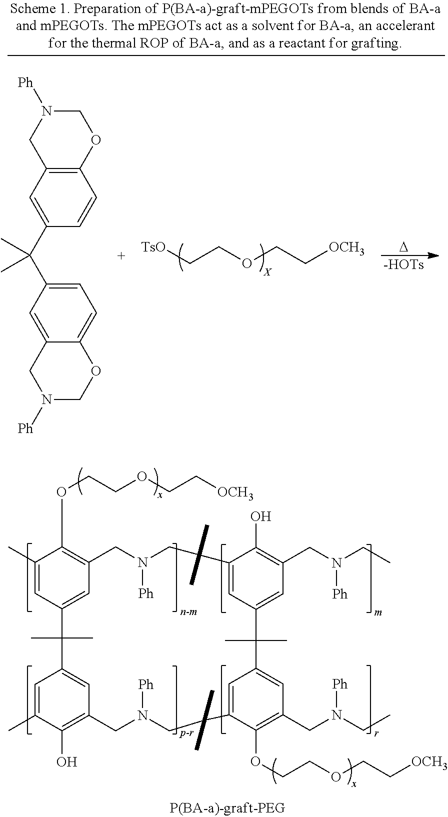

The bisphenol-A benzoxazine (BA-a) monomer was used as received from Hunstman. The methoxypoly(ethylene glycol) (mPEGOH₂₀₀₀; Mₙ 2000), methoxypoly(ethylene glycol) tosylate (mPEGOTs₂₀₀₀; Mₙ 2000), and methoxypoly(ethylene glycol) tosylate (mPEGOTs₉₀₀; Mₙ 900) were acquired from Sigma Aldrich. The methoxypoly(ethylene glycol) (mPEGOH₉₀₀; Mₙ 900) was used as received from Polymer Source. Acetone (99.7% purity) was used as received from Fisher Scientific. Chloroform (99.8% purity) was used as received from J.T. Baker. Deuterated dimethyl sulfoxide (99.9% purity) was used as received from Cambridge Isotope Laboratories, Inc. Neutral aluminum oxide was used as received from Acros Organics.

A Vega TS 5136MM scanning electron microscope (SEM), operated with an accelerating voltage of 15 kV, was used to capture micrographs of samples that were coated with a thin layer of gold and platinum to dissipate charge (nominal Au thickness ˜1 nm). Cured blend samples were prepared for SEM by fracturing fractioning the sample followed by a soak in chloroform for 30 minutes before drying in ambient conditions. A Nicolet iS10 FTIR spectrometer equipped with an attenuated total reflectance (ATR) accessory was used for FTIR studies (64 scans at 4 cm⁻¹ resolution per spectra). A Bruker AVANCE-III, HD 500 MHz NMR spectrometer was used to collect H-NMR spectra. Samples for NMR were prepared from cured materials that were soaked in DMSO-d6 for 30 minutes. A PANalytical X'Pert Pro diffractometer using monochromatic Cu Kα radiation with a wavelength (λ) of 0.15418 nm was used for X-Ray Diffraction (XRD) studies. XRD data fitting was conducted using a X′Pert HighScore Plus software package.

Preparation of Blends of Poly(ethyleneglycol) and N-Phenyl Bisphenol A Benzoxazine. The preparation of blends of either mPEGOH or mPEGOTs with N-phenyl bisphenol A benzoxazine (BA-a) followed a modified procedure outlined by Lü, H.; Zheng, S. Miscibility and phase behavior in thermosetting blends of polybenzoxazine and poly(ethylene oxide). Polymer 2003, 44, 4689-4698, incorporated herein by reference in its entirety. The general procedure for the preparation of BA-a blends was as follows: separate solutions of 1.45 g BA-a in 2.90 mL acetone and 2.05 g of either mPEGOH, mPEGOTs₉₀₀, or mPEGOTs₂₀₀₀ in 4.10 mL of acetone were prepared. Appropriate volumes from each solution were then combined to total 1 mL in a 10 mL round bottom flask. Each sample was dried in vacuo and then dried in a vacuum oven at 50° C. for 9 hours.

Thermal Analysis. A TA Instruments Q500 thermogravimetric analysis (TGA) instrument equipped with an inline Thermo Scientific iS10 Fourier Transform Infrared (FTIR) Gas Cell Spectrometer was used for TGA/TGA-FTIR experiments. TGA experiments were performed with a 1 min isotherm at 75° C. followed by a 25° C./min ramp from 75 to 30° C. and then a 10° C./min ramp from 30° C. to a minimum of 400° C. to analyze the weight changes during the melting and curing of BA-a, mPEGOH, mPEGOTs and their blends, and to determine any char yields. An isothermal transfer line (225° C.) was used to direct effluent from the TGA sample furnace to the FTIR spectrometer using dry nitrogen as a carrier gas (flow rate=90 mL/min). The background signal of FTIR spectra was collected from a 5 minute isotherm before and after this temperature program. FTIR spectra (consisting of 4 scans at 8 cm⁻¹ resolution and acquired at a 2.7 se interval) were corrected for background created from a time averaged signal (5 min) for the blank nitrogen carrier gas. A Perkin Elmer 4000 thermogravimetric analysis (TGA) instrument equipped with an inline Hiden QGA gas analysis mass spectrometer (MS) was used for TGA-MS experiments. Effluent from the sample was carried to the MS spectrometer via an isothermal transfer line (80° C.) and dry nitrogen gas flowing at 20 mL/min.

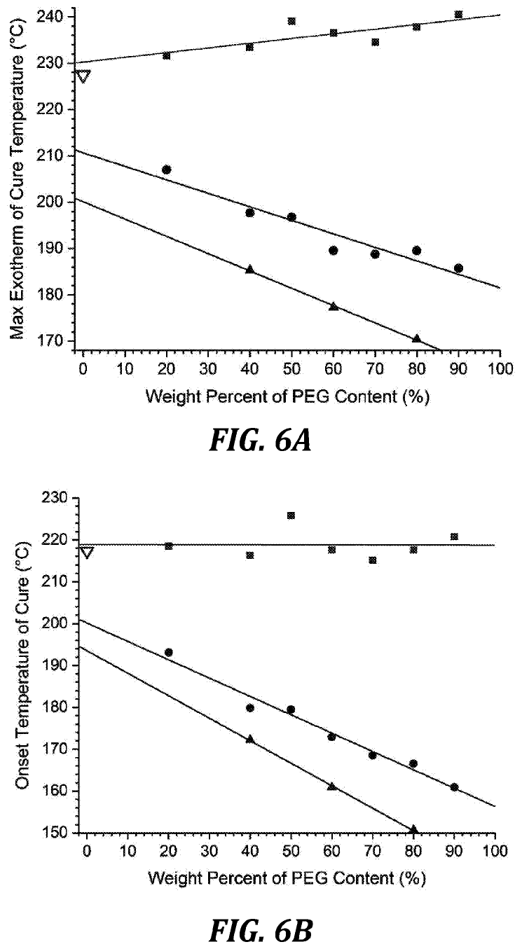

A TA Instruments Q100 differential scanning calorimetry (DSC) instrument was used for DSC-based cure studies on all samples. Blends were heated (in hermetically sealed DSC pans) for 3 min at the elevated temperature of 100° C. for mPEGOH blends and 50° C. for mPEGOTs blends. The pans were rapidly transferred to liquid nitrogen for a −200° C. quench then loaded into the DSC instrument. For cure studies, the samples were then scanned at 5° C./min from −70° C. to 75° C. to −70° C. to 75° C. (heat, cool, heat). The samples were heated and quenched as before and then scanned at 5° C./min from −70° C. to 260° C. to −70° C. to 260° C. Exothermic cure was estimated by integration of the exothermic peak using a tangential sigmoidal baseline (see FIG. 6A, 6B, and Table 2).

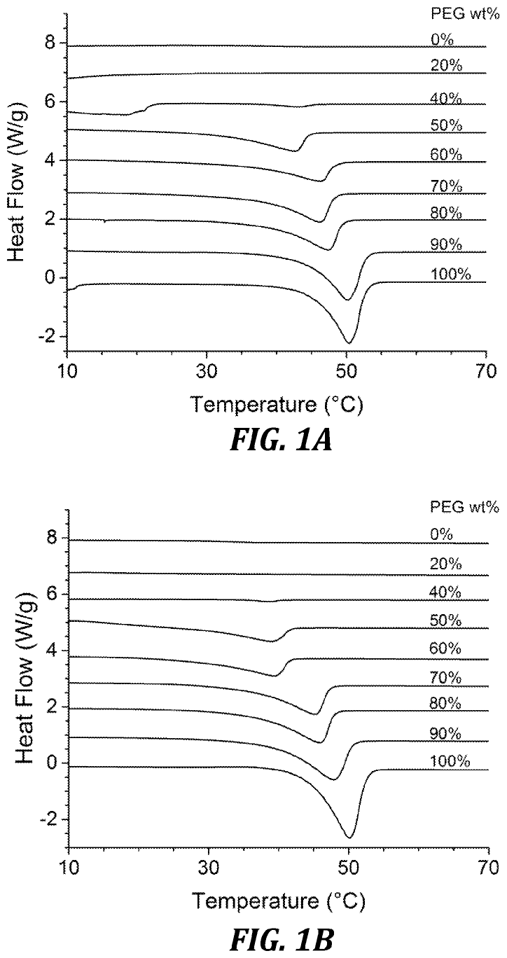

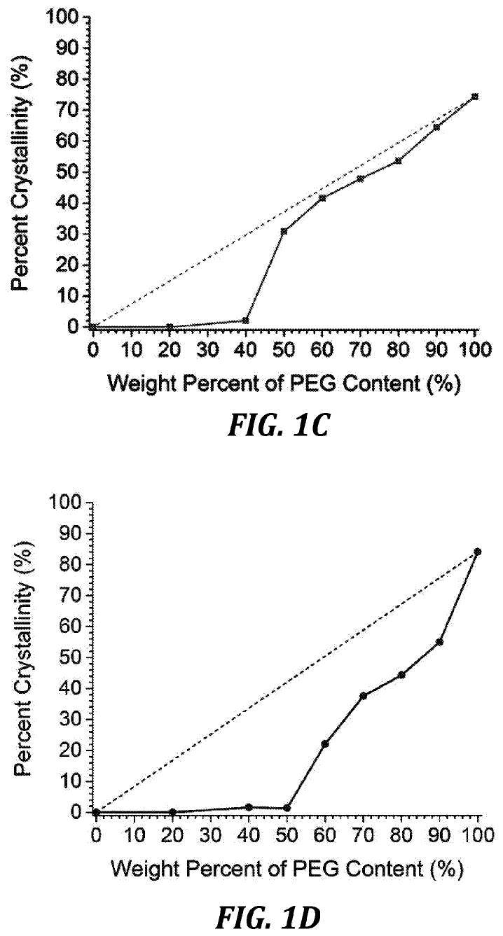

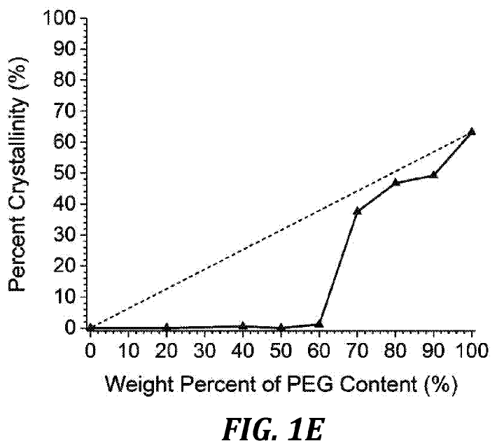

Miscibility of BA-a in mPEGOH and mPEGOTs and Crystallization. The miscibility of N-phenyl bisphenol A benzoxazine (BA-a) monomer in hydroxyl or tosylate end-group functionalized poly(ethylene glycol) (PEG) was investigated in order to better describe the polymerization, cross-linking and grafting reactions that can occur in their heated blends. Mixtures of BA-a with either methoxypoly(ethylene glycol) (mPEGOH₂₀₀₀; Mₙ=2000 Da) or methoxypoly(ethylene glycol) tosylate (mPEGOTs₂₀₀₀ or mPEGOTs₉₀₀; Mₙ=2000 and 900, respectively) were prepared by first co-dissolving in acetone, a common solvent for both BA-a and the PEG-based polymers. The solvent was evaporated at low pressure and temperature to create blended samples which were then sealed in hermetic vessels. The vessels were heated to melt any PEG-based crystallites and then thermally quenched in liquid nitrogen prior to thermal analysis by DSC. Shown in FIG. 1 are the heating curves for the BA-a, mPEGOH₂₀₀₀, and mPEGOTs₂₀₀₀ and the BA-a/mPEG blends.

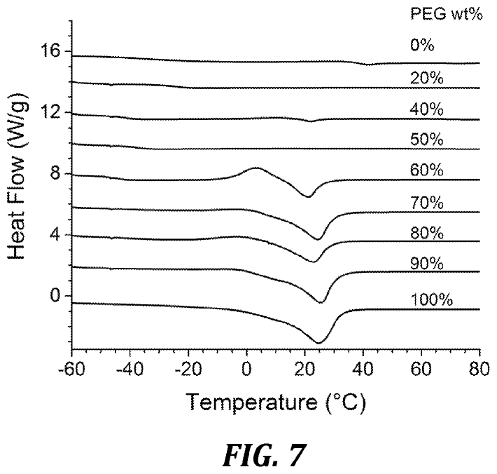

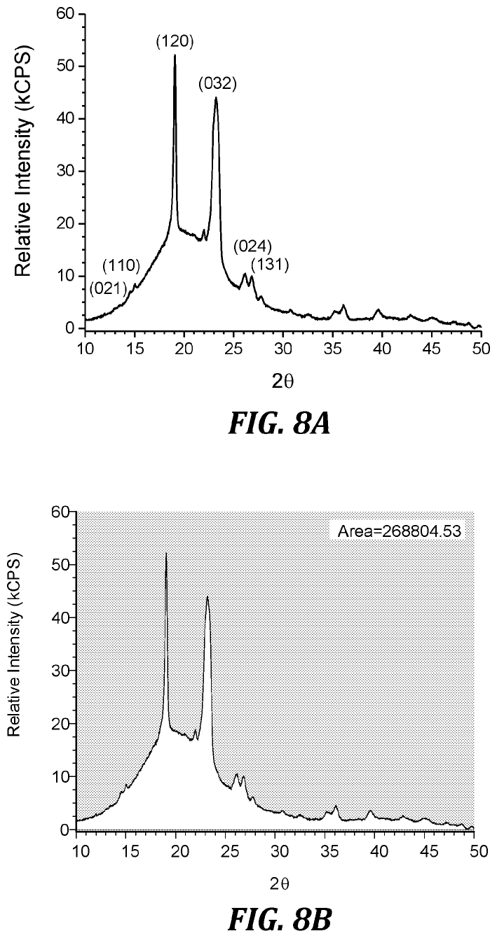

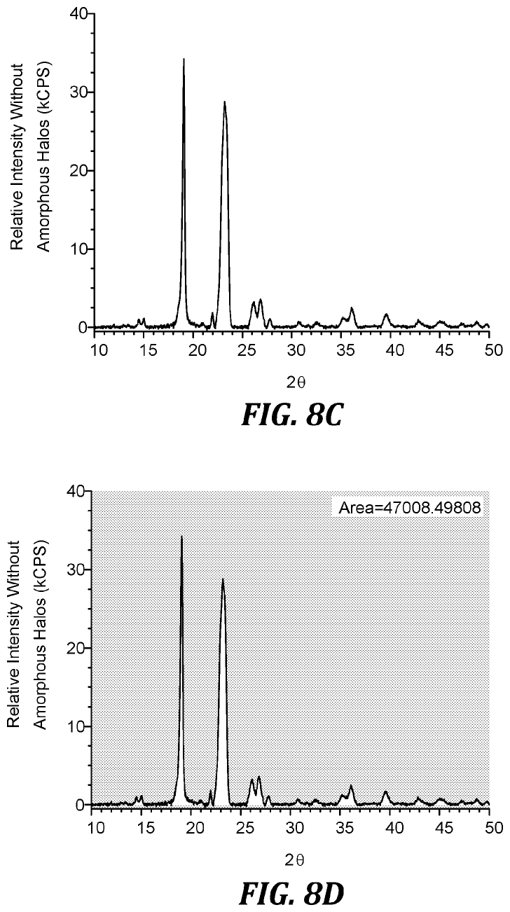

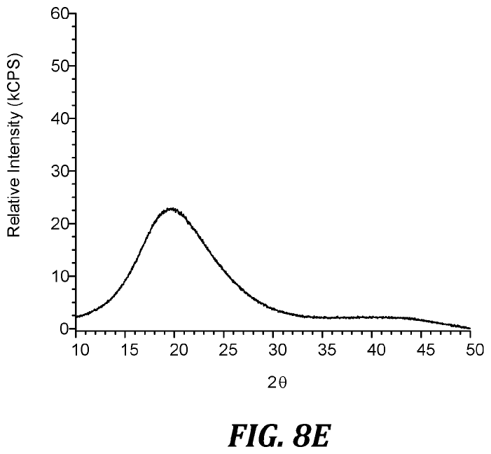

Similar heat curves for the mPEGOTs₉₀₀ series are found in FIG. 7. The PEG polymer used for samples in FIG. 1A was mPEGOH₂₀₀₀ and that for samples in FIG. 1B was mPEGOTs₂₀₀₀. The bottom-most curves in FIGS. 1A and 1B depict the thermograms for the isolated mPEGOH₂₀₀₀ and mPEGOTs₂₀₀₀. Each curve shows no evidence for an exothermic transition which confirms crystallization of the polymer occurred during the quench step. There was also little evidence for a glass transition temperature in the heating curves which shows that there is relatively little amount of amorphous content in these PEG samples. Each curve however depicts a notable endotherm with peak temperature values of 51° C. and 50° C. for the mPEGOH₂₀₀₀ and mPEGOTs₂₀₀₀, respectively. This endotherm corresponds to the melt transition (Tₘ) of PEG for which the Tₘ values agree with other PEG samples of similar molecular weight and structure. The enthalpy of melt (ΔHₘ) values for the mPEGOH₂₀₀₀ and mPEGOTs₂₀₀₀ were found to be 149 and 165 J/g, respectively, and are also in agreement with similar molecular weight PEGs. The top-most heating curve in FIGS. 1A and 1B depict the thermograms for the isolated BA-a monomer and show no evidence for crystallization, melting or curing. A notable difference between the blends with mPEGOH₂₀₀₀ and mPEGOTs₂₀₀₀ was observed in the change of their respective ΔHₘ before and after cure of the benzoxazine content (Table 3). Generally the former series maintained similar ΔHₘ in two successive heat curves with the second heat cycle mostly characterizing the sample after a cure event has occurred. The latter series however had a significant decrease in the ΔHₘ of the PEG in the second heat curve with no endotherm observed at all for blends ≤50 wt. % mPEGOTs. An XRD study of a representative sample from the mPEGOH₂₀₀₀ series with 40 wt. % mPEG revealed intensity maxima at 20 angles of 14.5°, 15.1°, 19.1°, 23.2°, 26.1°, 26.8° and 32.5°, which, are due to diffraction from the (021), (110), (120), (032), (024), (131) and (114) planes in crystallized PEG, respectively (see FIGS. 8A-8E). The degree of crystallinity was determined from ratio of the area of the diffractogram that excluded amorphous halos with that of the total area for the diffractogram that included areas for diffraction from crystallites and halos from amorphous material. The degree of crystallinity of the cured 40 wt. % mPEGOH₂₀₀₀ sample is therefore 17%. The corresponding data for a representative sample from the mPEGOTs₂₀₀₀ series, one with 40 wt. % mPEG, revealed no diffraction pattern and only amorphous halos, which, confirms that the crystallization of the mPEGOTs₂₀₀₀ suppressed in this cured sample. Without wishing to be bound by theory, it is believed that a random grafting of the PEG onto the PBA-a network occurs in this series and is responsible for suppressing crystallization of the grafted PEG (vide infra) in the second heating scan in the DSC curves.

From the first heat cycle in the DSC curves for the BA-a/mPEGOH₂₀₀₀ blends, it was apparent that crystallization and melting of PEG was not impeded by the presence of BA-a monomer for blends with compositions that range from 40 to 100 wt. % mPEGOH. BA-a/mPEGOH₂₀₀₀ blends with an overall PEG content less than 40 wt. % exhibited complete suppression of the melt endotherm and are therefore comprised of a majority amorphous state that hosts the dissolved BA-a. Similarly, from the DSC curves for the BA-a/mPEGOTs₂₀₀₀ blends, crystallization and melting of PEG was not impeded by BA-a monomer when blends were formulated with the composition range of 50 to 100 wt. % mPEGOTs. BA-a/mPEGOTs₂₀₀₀ blends with an overall PEG content less or equal to 50 wt. % exhibit complete melt suppression. Similarly, BA-a/mPEGOTs₉₀₀ blends with an overall PEG content less or equal to 60 wt. % exhibited complete melt suppression. The similar blend range for miscibility between BA-a and mPEGOTs and mPEGOH suggested that the end-group of mPEGOTs was not important for compatibilizing the solute. Also evident in FIG. 1A was a significant decrease in the Tₘ of mPEGOH₂₀₀₀ with increasing BA-a content in its blends. The melting point depression in FIG. 1A was due to the thermodynamic effect of incorporating the miscible phase into a crystalline mPEGOH phase. There was less melt point depression in the BA-a/mPEGOH system. The lowest melt points of the mPEGOH₂₀₀₀ and mPEGOTs₂₀₀₀ in the BA-a-richest blends were found at 43 and 38° C., respectively.

The inhibition of crystallization can be better represented by plotting the theoretical percent of crystallization of the PEG component versus the blend composition. FIGS. 1C-1E show the percent of crystallization of the mPEG component versus the BA-a-blend composition for the mPEGOH₂₀₀₀, mPEGOTs₂₀₀₀, and mPEGOTs₉₀₀ series. The enthalpy of melt for a completely crystallized PEO has been reported as ˜205 J/g, and was used to determine the percentage of crystallization in each blend sample discussed above. The enthalpy of crystallization for the pure mPEGOH₂₀₀₀ was used to determine the degree of crystallization which corresponded to a value of 74%, a value that agrees with previous reports for similar PEG materials quenched from its melt state. The degree of crystallization for the pure mPEGOTs₂₀₀₀ and mPEGOTs₉₀₀ had values of 84% and 63%, respectively. The difference in molecular weight of the two polymers was responsible for the difference in the degree of crystallinity of the two mPEGOTs polymers. If the crystallization of PEG was not influenced by the inclusion of the BA-a solute, then the percent of crystallization should correspond to the product of the fractional content of mPEG in the blend and the enthalpy of melt for the pure mPEG. Each plot in FIGS. 1C-E reported this theoretical percent of crystallization as a dotted line. In the mPEGOH₂₀₀₀ series (FIG. 1C), the percentage of crystallinity in the polymer did not deviate significantly from the predicted value for blends with ≥50% polymer content. In this same series, there was a dramatic decrease in crystallinity from the expected trend when the content of BA-a was more than 60 wt. % which indicates a miscibility range for the polymer and the BA-a monomer. In the mPEGOTs₂₀₀₀ and mPEGOTs₉₀₀ series (FIGS. 1D and 1E, respectively), similar characteristics were found in the corresponding plots but with an important difference. The miscibility range, indicated by the break from trend of the high PEG content blends (excluding pure PEG), was wider. Generally, the crystallization of the polymer was uninhibited for blends that were greater than 70 wt. % mPEGOTs₂₀₀₀ or mPEGOTs₉₀₀.

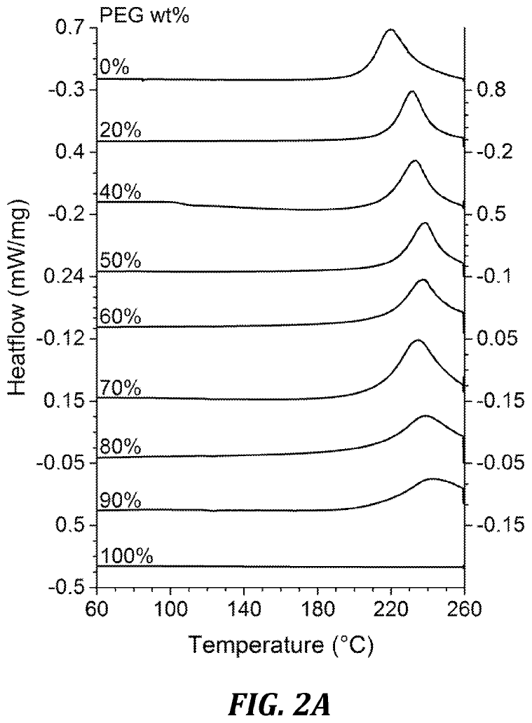

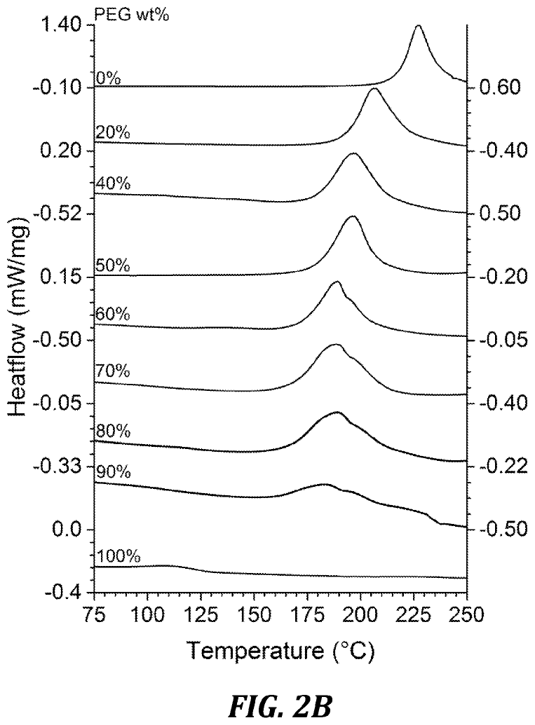

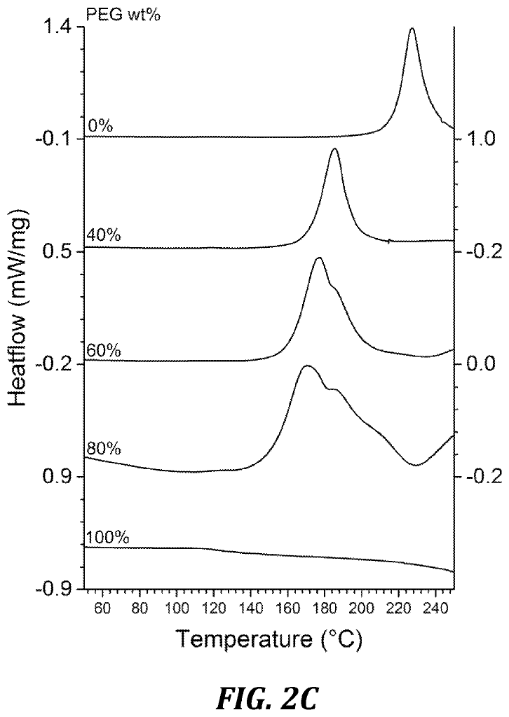

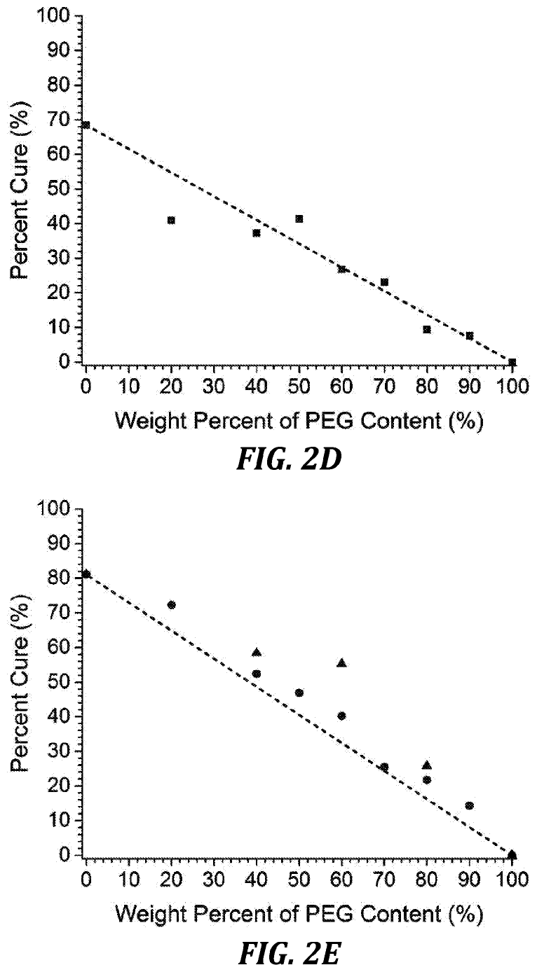

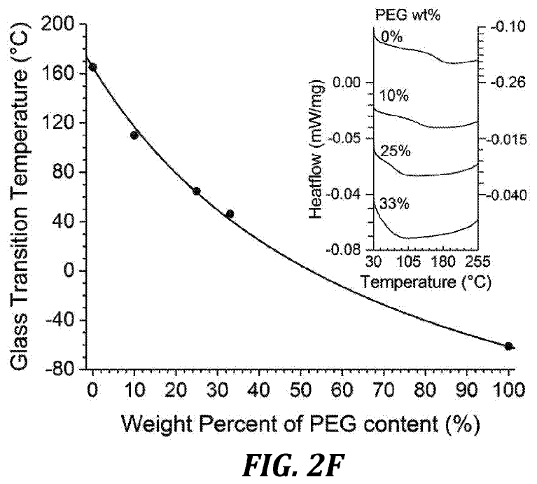

Cure of BA-a in mPEGOH and mPEGOTs blends. The thermal ring opening polymerization (ROP) of BA-a proceeded at elevated temperatures (>160° C.) and involved a cationic polymerization mechanism. A DSC cure study was conducted to understand the cure of the BA-a/mPEG blends. The temperature scan range of all the DSC runs were limited to 260° C. to minimize the influence of PEG degradation in this study. Shown atop in FIG. 2A is the DSC trace for pure N-phenyl bisphenol A benzoxazine (BA-a) which depicted an exotherm with a peak value and an onset value of 220 and 205° C., respectively. This exothermic signal corresponded to the energy of the thermal ROP reaction which was calculated to be 242 J/g. As PEG was introduced in the mPEGOH₂₀₀₀ blend series, the exotherm of the thermal ROP reaction for BA-a was found to shift to higher temperatures. Most curves were symmetric and monomodal with peak values in the range of 232-240° C. The onset of these peaks was also found to shift to higher temperatures as well (to 215-226° C.). The additional temperature for cure was likely a consequence of kinetic factors such as reduced heat transfer across the excluded and melted PEG phase to the monomer for polymerization. Inspection of the DSC curves for blends of BA-a with mPEGOTs revealed important distinctions. The DSC trace of the pure BA-a sample that was heated at 50° C., similar to the mPEGOTs blends, is shown atop in FIGS. 2B-2C. The enthalpy of the thermal ROP reaction was calculated to be 287 J/g for this sample with a peak and onset exotherm at 227 and 217° C. As mPEGOTs₂₀₀₀ was enriched in its BA-a blend series, the exotherm of the thermal ROP reaction for BA-a was found to decrease to lower temperatures with most peaks values in the range of 186-207° C. The onset of these peaks was also found to shift to lower temperatures (to 161-193° C.). The exotherm curves remained monomodal until the mPEGOTs₂₀₀₀ content exceeds a value of 50 wt. %. Based on the miscibility range inferred from FIG. 1B, the higher temperature exotherm shoulders would correspond to the cure of BA-a that was more closely associated with the once crystalline domains of the mPEGOTs. A beneficial interaction for polymerization existed between BA-a with mPEGOTs₂₀₀₀ and, since the p-toluenesulfonate group distinguished this polymer from the aforementioned one, without being bound by theory, it is believed that this end group was responsible for the reduction in energy required for initiation and propagation step in the polymerization of BA-a. In the three selected blends with mPEGOTs₉₀₀, the presence of the p-toluenesulfonate end group decreased the temperature of the thermal ROP reaction for the BA-a as well with peak temperatures between 170-185° C. and onset temperatures between 150-172° C., significantly lower than the mPEGOTs₂₀₀₀ blends. The blends with high mPEGOTs₉₀₀ content at 60 and 80 wt. % exhibit bimodal exotherms similar to the mPEGOTs₂₀₀₀ blends.

The standard enthalpy of cure for the thermal ROP reaction for the BA-a was 354 J/g. The exothermic signal for the exotherm atop in FIG. 2A corresponded to a percent cure value of 68% under the conditions used for the in-situ cure in the DSC. If the polymerization or cure reaction of BA-a into pure PBA-a was not influenced by the inclusion of the PEG, then the percent of cure should correspond to the product of the fractional content of BA-a in the blend and the enthalpy of cure for the pure BA-a. The dotted lines in FIGS. 2D-2E show these theoretical percent of cure predictions. The exotherm data in FIG. 2A was quantified by integration in order to estimate and report the enthalpy of cure for BA-a in these blends (see data in FIG. 2D). As PEG was introduced in the mPEGOH₂₀₀₀ blend series, the percent of cure of the BA-a closely followed the theoretical curve, suggesting that the thermal ROP of BA-a largely proceeded in the mPEGOH₂₀₀₀ without complication or side reaction. This also suggested that the hydroxyl-terminus of the PEG-OH did not undergo any reaction with the BA-a or PBA-a under these conditions and would result in a phase-separated or interpenetrating network mixture of the PBA-a and mPEGOH₂₀₀₀. The corresponding data for the mPEGOTs₂₀₀₀ and mPEGOTs₉₀₀ blend series showed significant differences. The exothermic signal for the thermal ROP of BA-a atop in FIGS. 2B-2C represented a percent cure value of 81%. Likewise the exotherm data in FIG. 2B-2C was calculated by integration and the percent of cure was reported in FIG. 2E. As the PEG was introduced in this series, the percentage of cure into pure PBA-a deviates positively from the predicted trend, demonstrating that there is a more complete cure reaction and/or a secondary exothermic reaction. Chemical information gleaned from FTIR and H-NMR supports this conclusion. The amount of positive deviation from the theoretical BA-a cure trend line was maximized when the BA-a:mPEGOTs was highest and exhibited no crystallization of the PEG component (i.e. 50 and 60 wt. % mPEGOTs₂₀₀₀ and mPEGOTs₉₀₀ samples, respectively). The lower melt viscosity for the lower molecular weight mPEGOTs₉₀₀ favored higher exotherm values in this regime.

The Gordon-Taylor relation can be used to generate a metric that quantifies the miscibility arising from favorable intermolecular interactions between components in polymer alloys. This relation, shown in eq. 1, calculates the glass transition temperature of miscible polymer-polymer systems (T_{g,ab}), using glass transition values of the independent blend components (T_{g,a}) and (T_{g,b}), and the weight fraction of each polymer in the blend (Wₐ and W_{b}).

$${T}_{g , ab} = {T}_{g , a} + k \frac{{W}_{b}}{{W}_{a}} ( {T}_{g , a} - {T}_{g , b} ) \qquad 1 )$$

The constant, k, in the Gordon-Taylor equation is a fitting parameter that provides curvature to the T_{g,ab} versus blend composition curve and usually takes on values from 0-1. The k values increase as the number and strength of favorable intermolecular interactions increases in blend systems. Shown as an inset in FIG. 2F are the DSC curves for the PBA-a created from the thermal ROP of BA-a and selected cured PEG blends from the mPEGOTs₂₀₀₀ series. All samples were cured using a heating program consisting of 10° C./min scans from 25° C. to 260° C. to 25° C. to 260° C. A glass transition value for pure PBA-a of 165° C. was found. A glass transition value for pure PEG (Mₙ=2000 Da) is also included on the plot. PEG-rich blends (those with mPEGOTs₂₀₀₀>33 wt. %) were not included in the plot. The fit of the Gordon-Taylor equation to the T_{g} values of the cured blends in FIG. 2F was good (R² value of 0.998) and gave rise to a fit constant k equal to 0.41. This fitting value shows that there are favorable molecular bonding interactions between the PEG and the cured BA-a material.

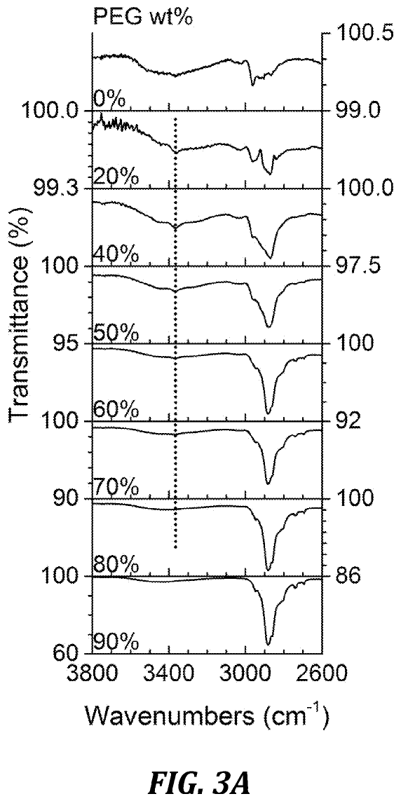

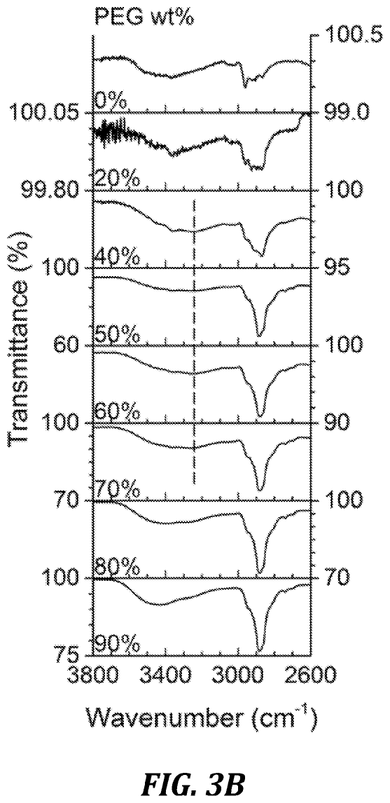

A FTIR study of cured blends of BA-a with mPEGOH₂₀₀₀ and mPEGOTs₂₀₀₀ was conducted in order to interrogate functional groups capable of intermolecular interactions as indicated in the Gordon-Taylor analysis above. The PBA-a that resulted from the thermal ROP of BA-a was expected to have phenol-based hydroxy residues that are capable of acting as a hydrogen bond donors. The oxygen atoms in the backbone of PEG are H-bond acceptors and, together, these groups lead to favorable intermolecular bonding interactions for improved miscibility to favor a more hybridized structure. Shown in FIG. 3 are the C—H and O—H stretch regions of the FTIR spectra of the cured samples from the BA-a/mPEGOH₂₀₀₀ series. The spectra atop in both FIGS. 3A and 3B display the FTIR data for pure PBA-a with signals for both sp³ CH₃ and CH₂ residues (ν_{CH3}: 2870-2970 cm⁻¹; ν_{CH2}, 2850-2925 cm⁻¹, respectively) and aromatic sp² CH residues (ν_{aromtic CH}: 3010-3050 cm⁻¹) as well as a broad signal for phenol hydroxyl groups (ν_{O—H phenol}: 3360 cm⁻¹). The broadness of the OH-stretch peak reflects a wide distribution of weakly hydrogen-bonded hydroxyl groups that are characteristic of phenolic polymers. As PEG was increased in content in the blend series for mPEGOH₂₀₀₀ (down the stack plot in FIG. 3A), the intensity of the peak associated the ν_{CH2} stretching mode of sp³ bonded CH₂ strengthened and evolved in accordance with the increase in the methylene content in the PEG loaded sample. The signal for the aromatic sp² CH residues (ν_{aromatic CH,}: 3010-3050 cm⁻¹) also became less prominent with increasing PEG content. The broad signal associated with phenol hydroxyl groups became reduced with increasing PEG content and, displayed its maximum signal at a near constant wavenumber of 3360 cm⁻¹, which indicates a similar chemical distribution of polymeric hydroxyl groups regardless of blend composition. Only the sample with 90 wt. % PEG showed a shift in this hydroxyl stretch, showing the peak at 3424 cm⁻¹, which indicates reduced hydrogen bonding. Therefore, as a whole, PBA-a phenol groups are weakly associated with the PEG constituent across the entire compositional range of samples. Inspection of FIG. 3B showed some similarities and differences in cured samples from the mPEGOTs₂₀₀₀ series. In similarity to the aforementioned series, as the mPEGOTs₂₀₀₀ increases in these samples (down the stack plot in FIG. 3B), the transmission peak associated ν_{CH3} sp³ bonded CH₃ decreased in favor of increased ν_{CH2} signal for sp³ bonded CH₂. Similarly, the ν_{aromatic CH} signal for the aromatic sp² CH residues became less prominent with increasing PEG content. In contrast however, the hydroxyl stretching band shifted to a lower frequency of about 3250 cm¹ when the mPEGOTs₂₀₀₀ content was between 40-70 wt. %. The samples with 80 and 90 wt. % mPEGOTs₂₀₀₀ also showed this low frequency peak but only as a prominent shoulder on the overall hydroxyl peak. The dotted and dashed lines in FIGS. 3A and 3B tracked the maximum signal of the hydroxyl stretching mode for the mPEGOH and mPEGOTs series. The low frequency hydroxyl stretch mode was associated with hydrogen bonding interactions between ring-opened BA-a and the PEG chain and indicated a hydroxyl hydrogen bond interaction that was much stronger in mPEGOTs blends than that found in the pure PBA-a polymer (atop in FIG. 3) or in mPEGOH blends. A molecular scale integration of the PBA-a and mPEGOTs occurred in this series of blends.

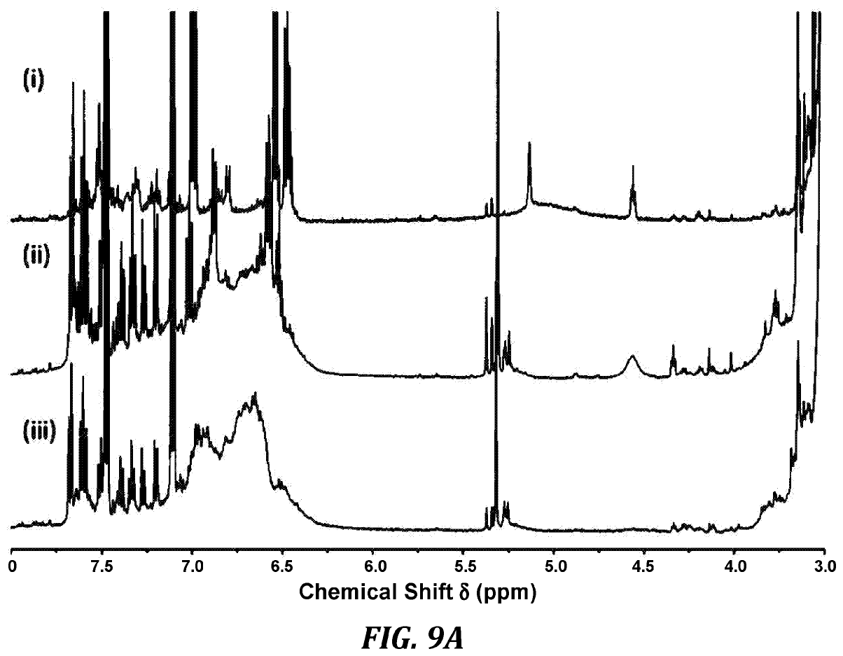

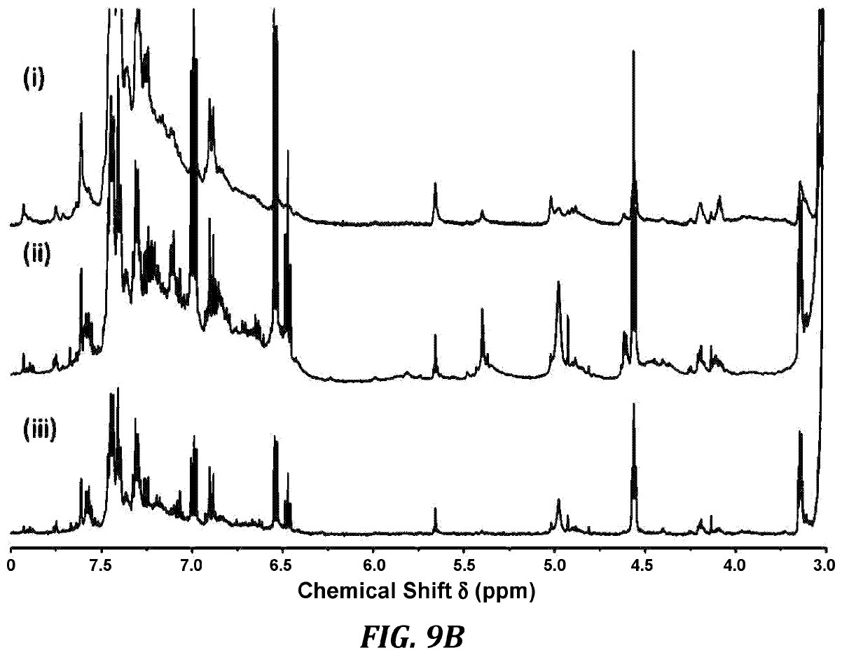

H-NMR spectra of extractable polymer segments from cured blends (FIG. 9) confirms that the molecular structure of the PBA-a network generated in mPEGOTs blends was enriched in phenolic groups. Previous work has shown that when BA-a was cured with a short cure time or without a catalyst, the resulting PBA-a polymer consisted of a more phenoxy-rich structure with more methylene signals in the 4.3-5.0 ppm range in the H-NMR spectrum. A prolonged cure time or the introduction of a Lewis acid catalyst accessed the more phenolic-rich structure typically evidenced by a greater intensity of methylene peaks in the 3.5-4.0 ppm range. The aromatic region of the spectrum also became more well-defined with greater intensity to fewer peaks in the 6.5-7.0 ppm range for phenolic-rich PBA-a. Based on the three broad methylene peaks at 4.5, 5.0, 5.4 ppm in FIG. 9A, the PBA-a network that resulted from the thermal ROP of dissolved BA-a in mPEGOH appeared to be enriched in the less thermodynamically stable phenoxy molecular structure. Conversely, the lack of significant peaks in the 4.3-5.0 ppm range and the greater intensity associated with a shoulder peak (δ=3.8 ppm) on the foot of PEG methylene peak (δ=3.5 ppm) in the H-NMR for BA-a/mPEGOTs blends (FIG. 9B) indicated a significant enrichment in the more thermodynamically stable phenolic structure in these crosslinked PBA-a networks. The appearance of more well-defined aromatic peaks at 6.5, 6.7 and 6.9 ppm in the spectra of these cured blends also supports this structural assignment.

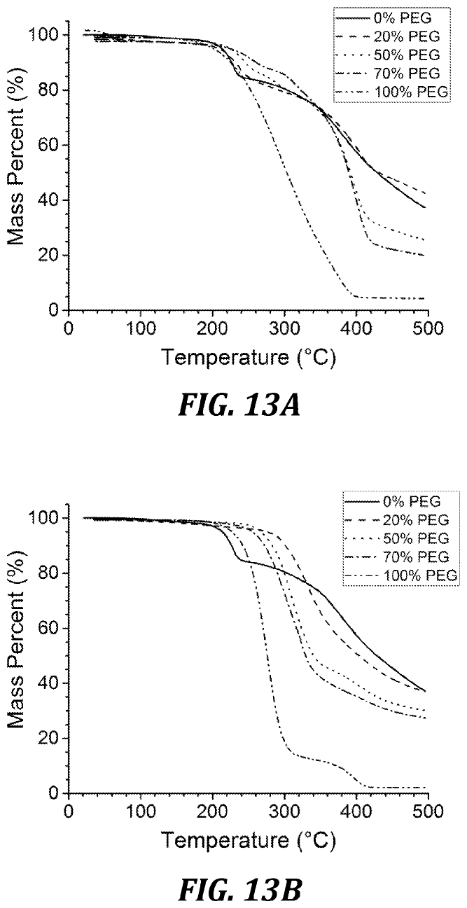

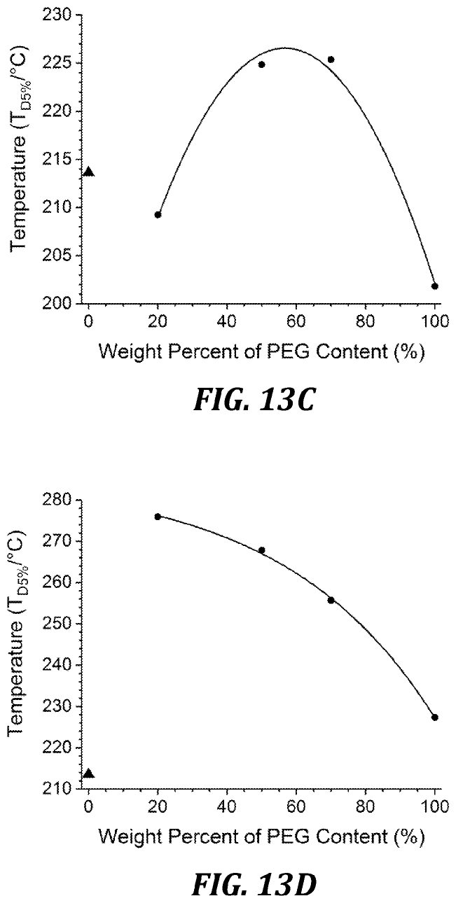

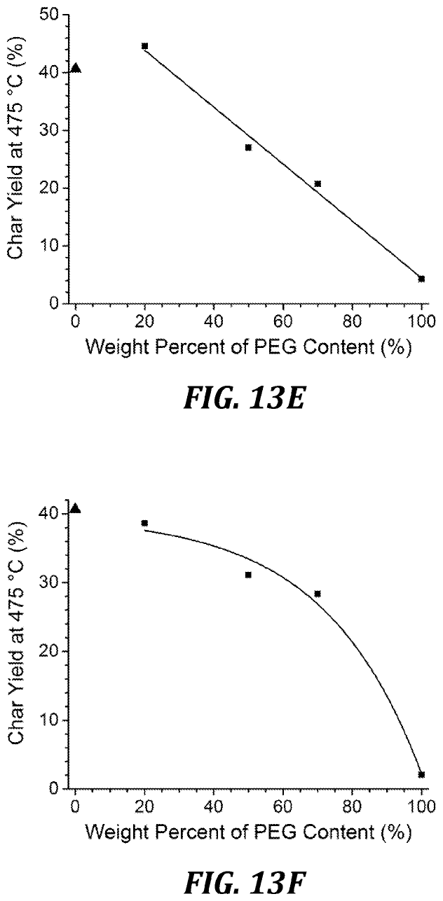

The excess exothermic energy in the cure DSC study and the high level of molecular mixing for the cured BA-a/mPEGOTs series suggested that a reaction occurs between the two components during the thermal cure process. In order to probe at the nature of the chemical reaction between BA-a/mPEGOTs and at the thermal stability of the resulting materials, Thermogravimetric-Fourier Transform Infrared (TGA-FTIR) and Thermogravimetric-Mass spectroscopy (TGA-MS) studies were performed. Lower molecular weight mPEGOH and mPEGOTs (both with Mₙ=900 Da) were selected in order to better emphasize signal associated with the end groups of the polymers. FTIR intensity maps for temperature vs. wavenumber of the TGA-FTIR data sets are included in FIGS. 9-12. The corresponding TGA curves are also shown in FIG. 13. Generally, TGA curves for the BA-a/mPEGOH₉₀₀ samples showed two main weight loss events with onset temperature values of ˜220° C. and ˜370° C. Those with more PEG content showed more pronounced weight loss at 370° C. TGA curves for the BA-a/mPEGOTs₉₀₀ showed one major weight loss event at 380° C. Interestingly, the thermal stability of the cured blends increased as mPEGOH and mPEGOTs were introduced. This thermal stability was reflected in the plots for the temperature for 5% weight loss (T_{D5%}) versus the PEG content (FIGS. 13C-13D). The increase in the thermal stability in the mPEGOH series was modest, with a maximum value in FIG. 13C of 225° C. for the 70 wt. % mPEGOH sample which was 12° C. above that of pure PBA-a. Remarkably, the influence of the mPEGOTs on the thermal stability was much more pronounced. Cured blends were generally much more thermally stable than those from the mPEGOH series and, notably more stable than pure PBA-a. As the mPEGOTs content was set from 0 to 20 wt. %, the T_{D5%} increased by ˜50° C. (see FIG. 13B). This metric for thermal stability also appeared to follow a relatively good correlation to the composition of the blend, where mPEGOTs content in excess of 20 wt. % adjusted the thermal stability to a lower value but still in excess of that of pure PBA-a. The char yield at 475° C. for pure PBA-a was 41%. The char yield of BA-a/mPEGOH blends appeared to follow a decreasing linear trend for the samples with greater than 20 wt. % mPEGOH. Only the sample with 20 wt. % mPEGOH blend exhibited a char yield in excess of that of the pure PBA-a. The 475° C. char yield of BA-a/mPEGOTs blends appeared to decrease with increasing mPEGOTs but did so with a non-linear dependence on the mPEGOTs content. Most of the tested blends exhibited 475° C. char yield values (28-39%) close to that of the pure PBA-a. Blends from the mPEGOTs favor good thermal stability even in the high temperature range past the main mass loss event. Without wishing to be bound by theory, it is believed that the increased thermal stability of the BA-a/mPEGOTs blends arises from the increased phenolic structure deduced by the H-NMR data. The less thermodynamically stable phenoxy linkages that were enriched in the BA-a/mPEGOH blends led to a greater content of labile bonds and increased reactivity at lower temperatures and consequently reduced mass during the thermal stress of the TGA analysis.

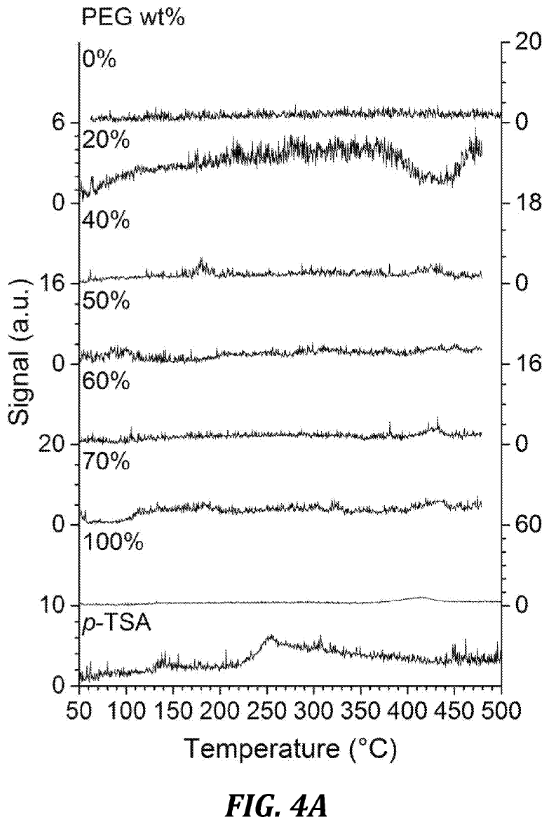

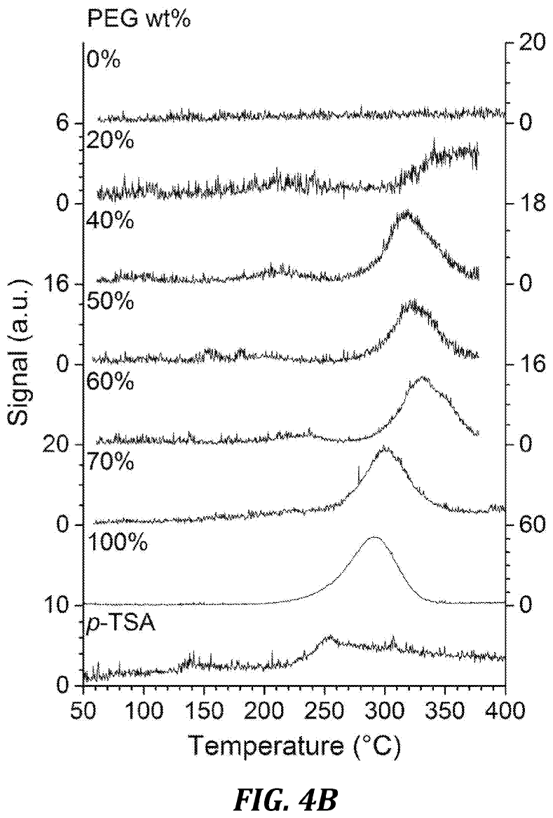

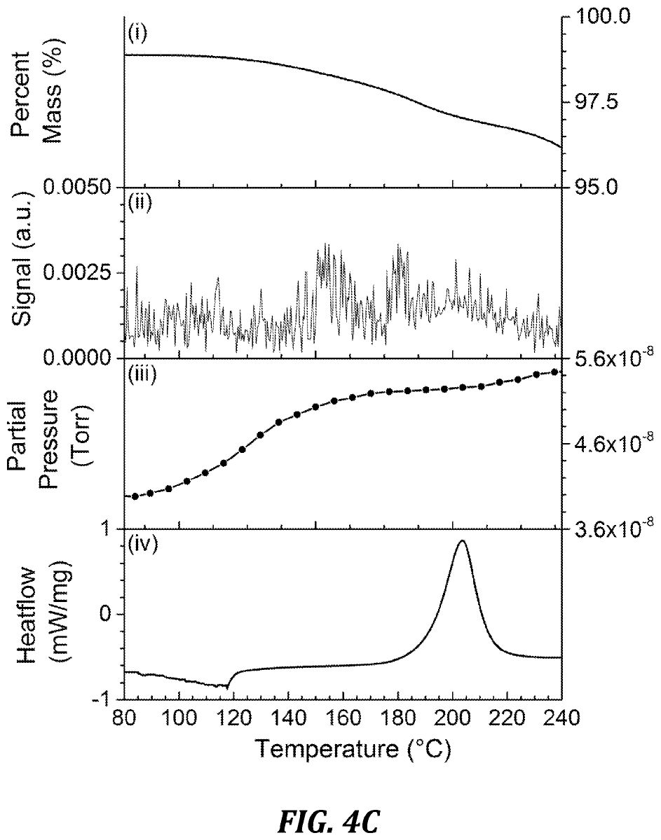

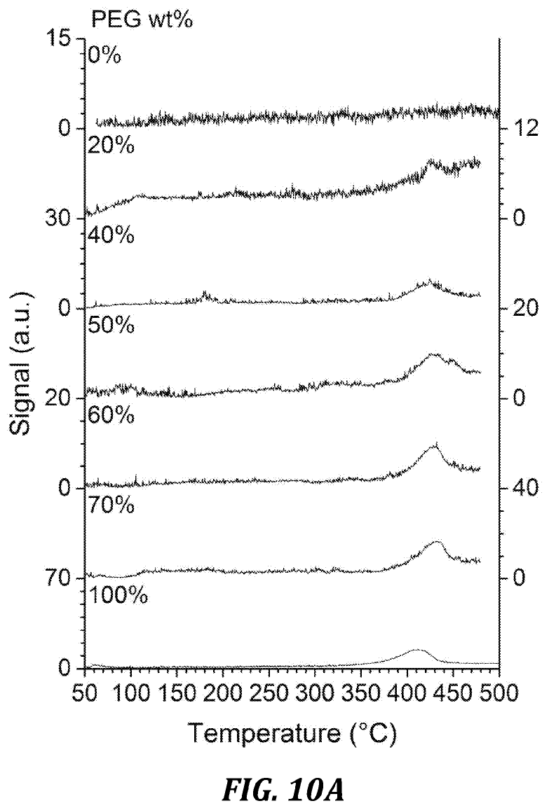

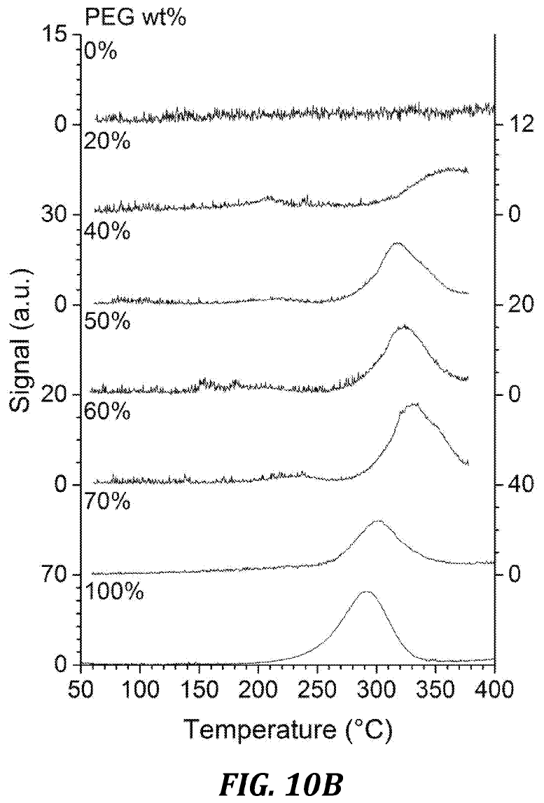

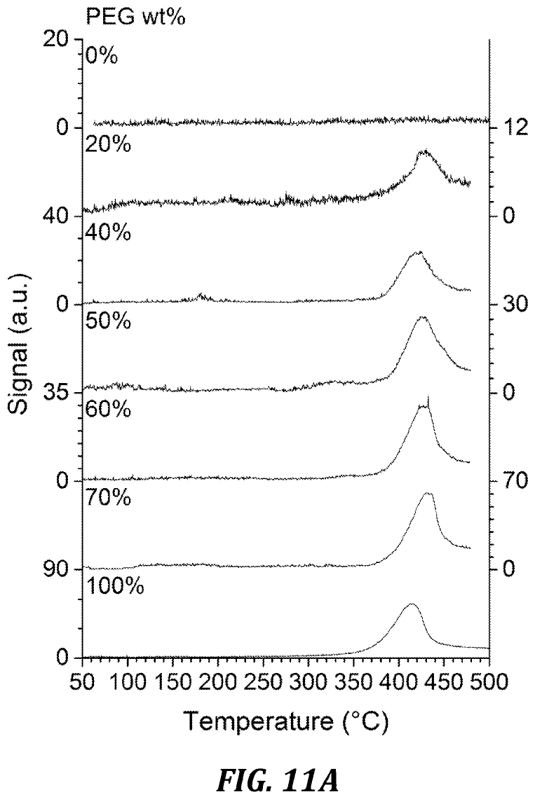

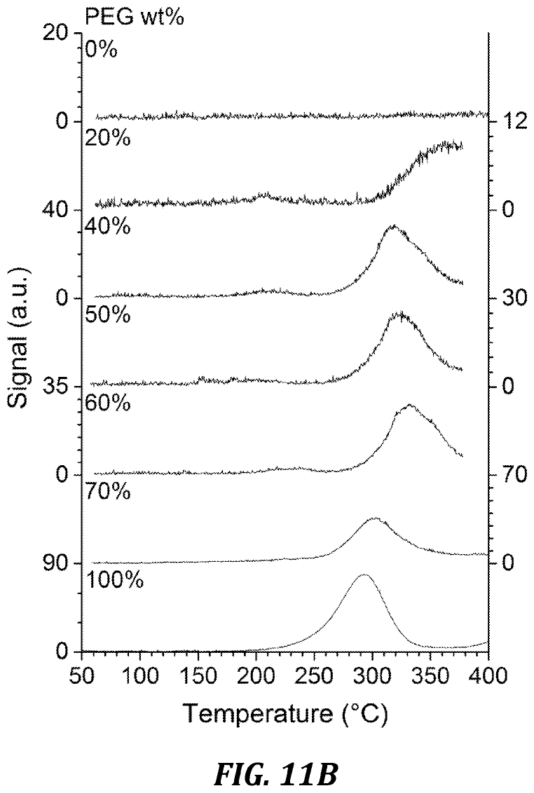

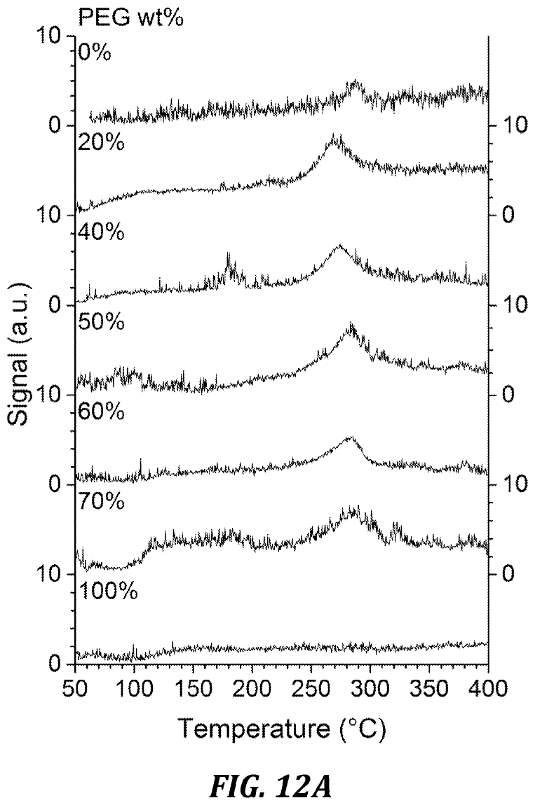

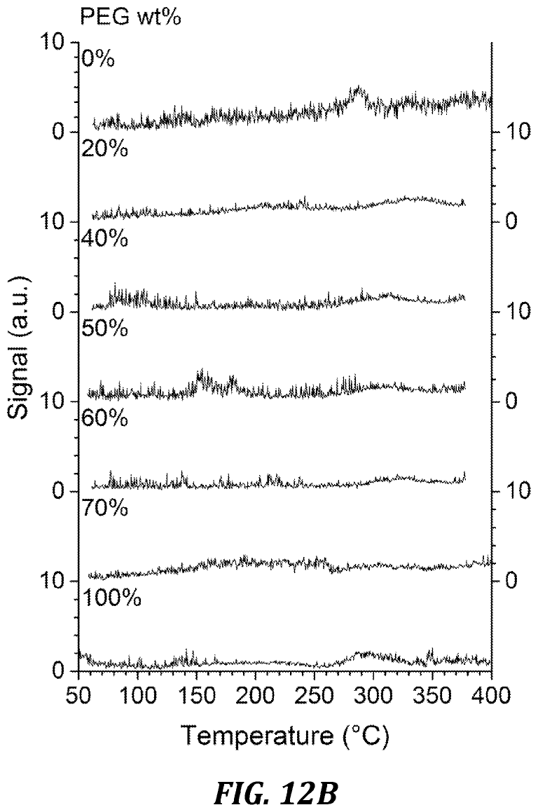

FTIR intensity tracer plots that relate the intensity of absorbance for vibrational modes associated with volatiles from the samples were created and reported in FIGS. 10-12 and FIGS. 4A, 4B, and 4C(ii). In the mPEGOH₉₀₀ series, the tracer plots for the ν_{CH3} (2973 cm⁻¹) all showed that a methyl-containing volatile was liberated at approximately 380° C. with its peak intensity at 430° C. (with the exception of the pure PBA-a and mPEGOH constituents, see FIG. 10A). Similarly, an ether-containing volatile (detected with a ν_{CO} at 1136 cm⁻¹) was liberated with similar onset and peak temperature values for blends. This peak shifted to lower temperature for the mPEGOH and was not present in the pure PBA-a (see FIG. 11A). Volatiles associated with a ν_{aromatic CH} at 3048 cm⁻¹ were liberated from mPEGOH blends with an onset and peak temperature of 250° C. and 280° C., respectively, (see FIG. 12A). Pure PBA-a showed a similar volatilization of aromatic compounds bearing sp² CH units suggesting an excellent retention of carbon at high temperatures, a high char yield characteristic that was well-documented for P(BA-a)s. No significant signal for sulfonate containing groups (that would be identified by a ν_{S—OH} stretch at 885 cm⁻¹, see FIG. 9A) was found for pure PBA-a, mPEGOH₉₀₀ or their blends. Given the phase separated state that was indicated in the DSC study of the BA-a blends with mPEGOH₂₀₀₀ and the fact that PEG was known to degrade at ˜360° C., without wishing to be bound by theory, it is believed that additional reduced thermal stability can arise from a higher surface area PBA-a material that is generated when phase-separated PEG is volatilized from the blends. Significant loss of mPEGOH was confirmed with the ether signal in the TGA-FTIR in FIG. 11A which aligns with this hypothesis.

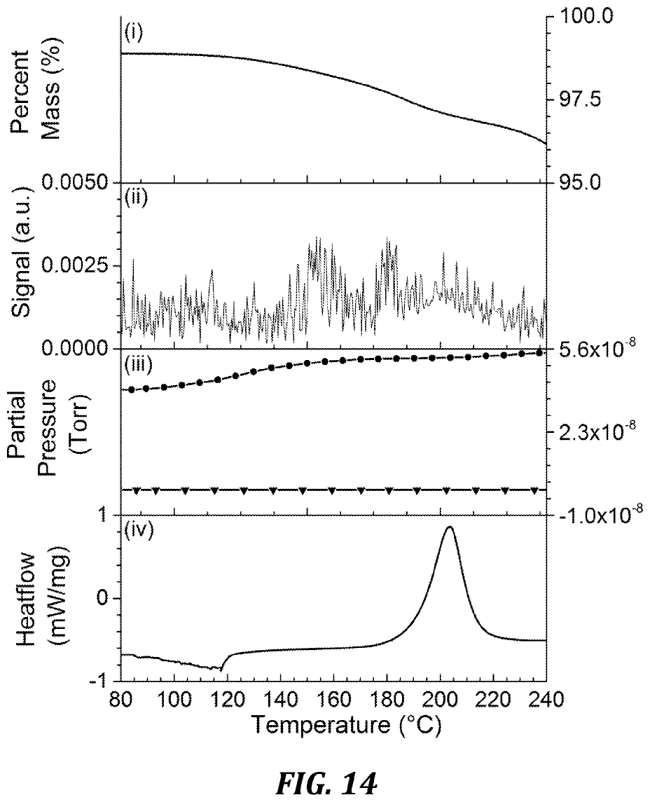

The TGA-FTIR study for the mPEGOTs₉₀₀ series showed differences in its tracer plots. In general, signals for volatiles with methyl, ether and sulfonate residues were detected. Furthermore, samples that demonstrated high miscibility of BA-a for mPEGOTs (40-60 wt. % mPEGOTs) showed signals for these volatiles at higher temperatures than those that were phase separated (see FIG. 1C). Particularly interesting was the lack of signal for volatiles containing aromatic groups. This result indicated a better retention of sp² carbon and explained the increased fractional char yield for mPEGOTs samples (see FIG. 13 for a comparison of the TGA plots for blends for 40-60 wt. % mPEGOH with 40-60 wt. % mPEGOTs). The tracer plots for the S-OH stretch at 885 cm¹ vs. temperature for BA-a blends with mPEGOH₉₀₀ and mPEGOTs₉₀₀ are reported in FIGS. 4A and 4B, respectively, and also include the analogous data acquired from the TGA-FTIR for p-toluene sulfonic acid (p-TSA). In the latter, the onset for the major signal was 220° C. and showed a maximum intensity at 250° C. A weak signal for the vs-OH, beginning at ˜110° C., largely coincided with the onset for the first mass loss event found by TGA. The coinciding signals for the major p-TSA flux and that of the pure mPEGOTs₉₀₀ strongly suggested that heating the tosylate capped mPEG led to a scission of the p-TSA group from the polymer allowing for liberated p-TSA to act as a catalyst for the thermal ROP of BA-a. The detection of the S-OH signal for miscible blends at higher temperatures suggested that the p-TSA also has additional intermolecular interactions in the BA-a blends which would demand additional energy for volatilization. The hypothesis for generating p-TSA from heated mPEGOTs was confirmed in a TGA-MS study of the BA-a/mPEGOTs₉₀₀ blend where the mPEGOTs content was 50 wt. %. Shown as plot iii in FIG. 4C is the partial pressure signal associated with the tolyl molecular ion fragment (m/z=91 Da) of volatilized p-TSA as the temperature was ramped. A control experiment confirmed that this signal did not originate from curing BA-a into PBA-a (see FIG. 14). Co-plotted with this data are the corresponding TGA, DSC and vs-OH tracer data. Plot iii in FIG. 4C showed that two fluxes of p-TSA evolved from the sample, where the initial rise in signal for the tolyl fragment began at −100° C. and plateaued by a temperature of 180° C., a temperature range which largely coincides with the onset for the first mass loss event found by TGA. A weak qualitative signal for the vs-OH in the tracer plot was found near this temperature. The secondary MS signal for the tolyl fragment initiated at ˜200° C. which closely aligned with the maximum in the cure exotherm as found in the DSC (see plot iv in FIG. 4C). These two fluxes of evolved p-TSA suggested that two mechanisms were involved in the generation of the molecule with the lower temperature event associated with generation of initiators and the higher temperature event being strongly associated with the ROP of BA-a.

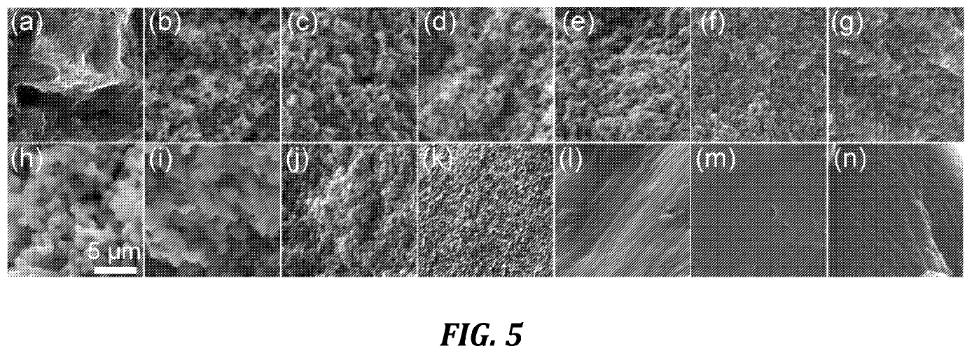

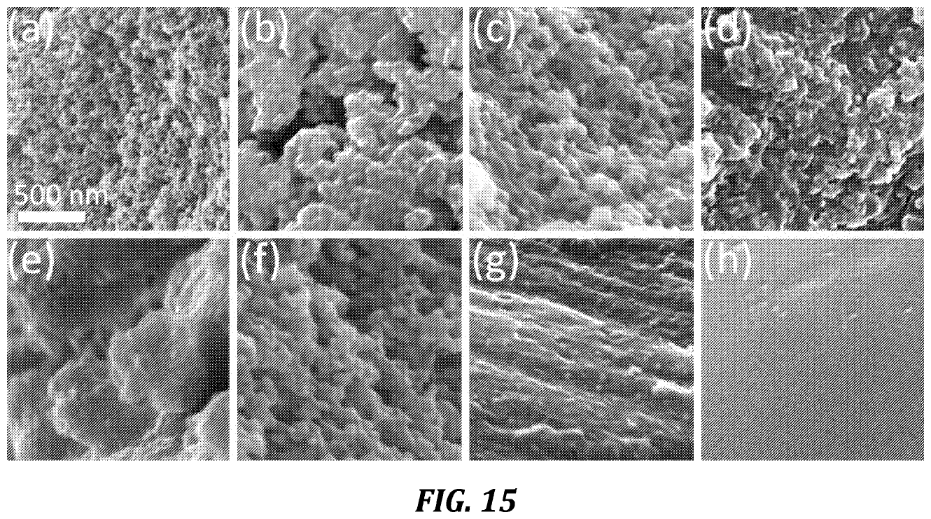

Scanning electron microscopy (SEM) was used to investigate the microstructure of the cured BA-a blends (see FIG. 5). Following a cure condition (identical to that used in the DSC study), the mPEGOH₂₀₀₀ and mPEGOTs₂₀₀₀ samples were extracted with chloroform and then cooled for cleavage prior to the SEM study (high resolution SEM images are found in FIG. 15). All samples from the mPEGOH₂₀₀₀ series showed a highly porous morphology. The sample that was weighted the most with mPEGOH₂₀₀₀ (90 wt. %) exhibited the most heterogeneous structure with large void spaces that were greater than 10 μm in size. The void space in these samples arose from regions that were previously occupied by the mPEGOH₂₀₀₀ material that was extracted by the chloroform solvent. As the mPEGOH₂₀₀₀ content is decreased (FIGS. 5(a-g)) the void space was observed to decrease and the uniformity in the PBA-a phase increased. The ability to tune the void content in the cured PBA-a from blends with mPEGOH₂₀₀₀ can be used for tuning transparency, macroscopic density, and strength characteristics in cured macroscopic parts made from these blends.

Indeed, all cured mPEGOTs/PBA-a materials are transparent yellow-orange materials while those of cured mPEGOH/PBA-a are orange and opaque. The PBA-a-richest samples tend to adopt a fused particle like morphology with complex and wide interconnections. There is little evidence for any structural implications that would be from the miscible condition that was observed for uncured samples with <40 wt. % mPEGOH₂₀₀₀ (FIG. 1A). The SEM data therefore agree with the conclusion from the FTIR study that the polymerization of BA-a monomer leads to a PBA-a that is phase-separated from the mPEGOH₂₀₀₀ polymer. For cured samples with a completely suppressed crystallization of mPEGOTs₂₀₀₀ (FIG. 1B) and a high content of hydrogen-bonded hydroxyl groups (<50 wt. % mPEGOTs₂₀₀₀), a highly continuous and smooth morphology was indicated in the SEM images (see FIG. 5 (1-n)). The microstructure of cured samples where there was crystallization of mPEGOTs₂₀₀₀ resulted in a porous microstructure, reminiscent of that in the mPEGOH₂₀₀₀ series. This structure is highly uniform, where sphere-like nanoparticles of PBA-a become larger and more well-defined as the mPEGOTs₂₀₀₀ content increases (varying from ˜50 to ˜500 nm in diameter for the 60 to 90 wt. % mPEGOTs samples, respectively). A rather abrupt transition in particle size was found between samples that were 80 and 70 wt. % mPEGOTs₂₀₀₀ which coincided with the threshold ratio needed to suppress some of the crystallization of the mPEGOTs₂₀₀₀ (FIG. 1). This in combination with the data from the SEM, DSC, and FTIR studies support a more hybridized and bonded chemical structure for cured BA-a/mPEGOTs samples. A graft structure was assigned to P(BA-a)-g-PEG samples.

Mechanistic aspects for the cure of BA-a/mPEGOTs blends. For the polymerization of the BA-a/mPEGOTs blends, a pegylated cationic oxonium species would result from the nucleophilic attack by the oxygen site of a BA-a monomer at the tosylate end group of the mPEGOTs (see Pathway A in Scheme 2). This oxonium would establish an equilibrium with the ring-opened iminium species, both of which can initiate ROP of other BA-a monomers. In the alternative Pathway B, the nitrogen center of the BA-a monomer acts as the initial nucleophile and a pegylated ammonium species is afforded by reaction with the mPEGOTs. This specie can undergo a secondary nucleophilic attack by another BA-a molecule to produce the same pegylated cationic oxonium species discussed in Pathway A. Thermal dissociation of bulky alkyl p-toluenesulfonates can produce free p-toluenesulfonate (⁻OTs) and cationic species such as the cyclohexyl secondary carbocation for initiation of BA-a. The thermal dissociation of the mPEGOTs was shown as Pathway C in Scheme 2 and would produce a primary carbocation by this mechanism. Without wishing to be bound by theory, it is believed that the high reactivity of primary carbocation makes pathway C unlikely, and favors the generation of the cationic initiators by either Pathway A or B. Both of these routes lead to free ⁻OTs as a byproduct in the early stages of the thermal cure of the BA-a/mPEGOTs blends. The TGA-MS experiment described above detected this early flux of ⁻OTs (i.e. a tolyl molecular fragment) which further supports a thermal initiation of BA-a by its nucleophilic attack on the mPEGOTs.

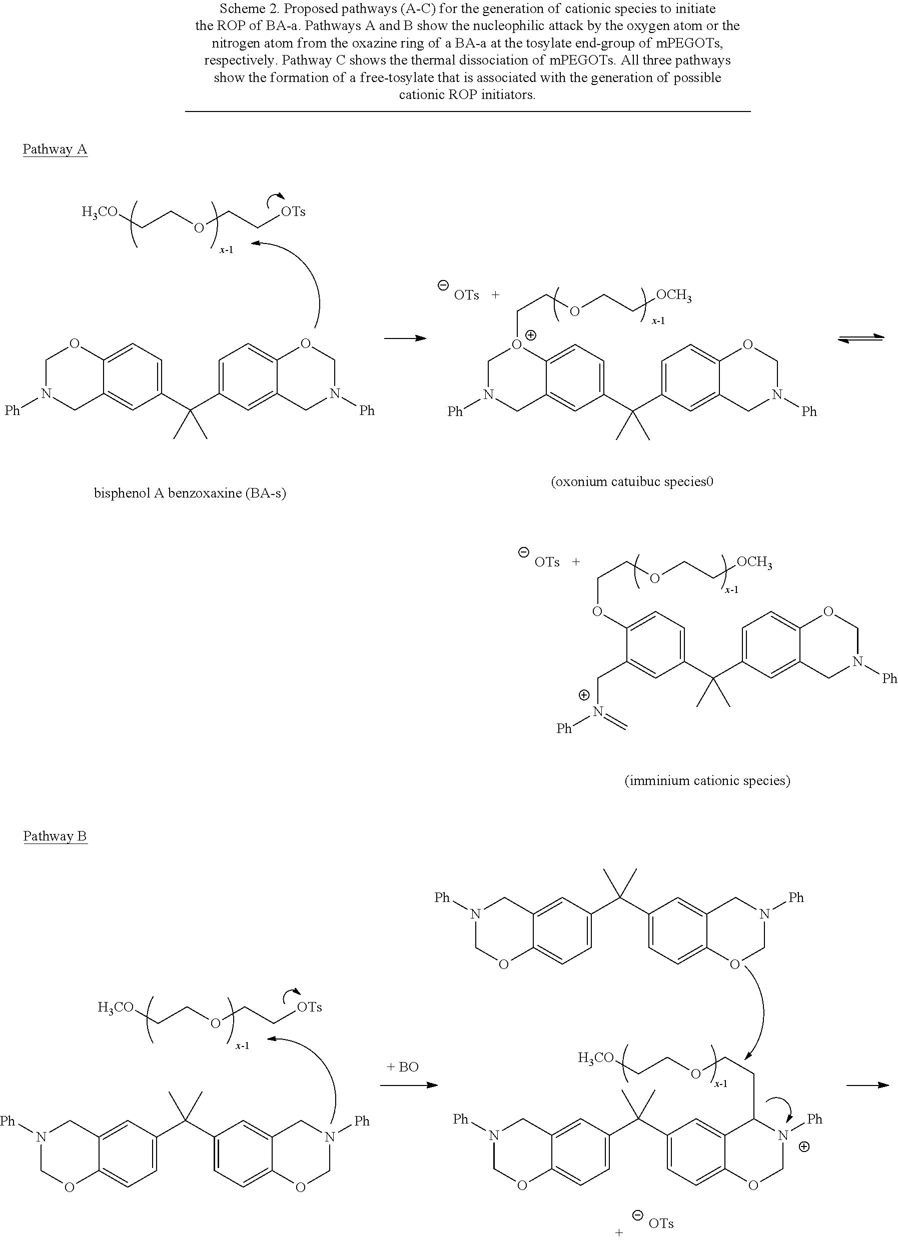

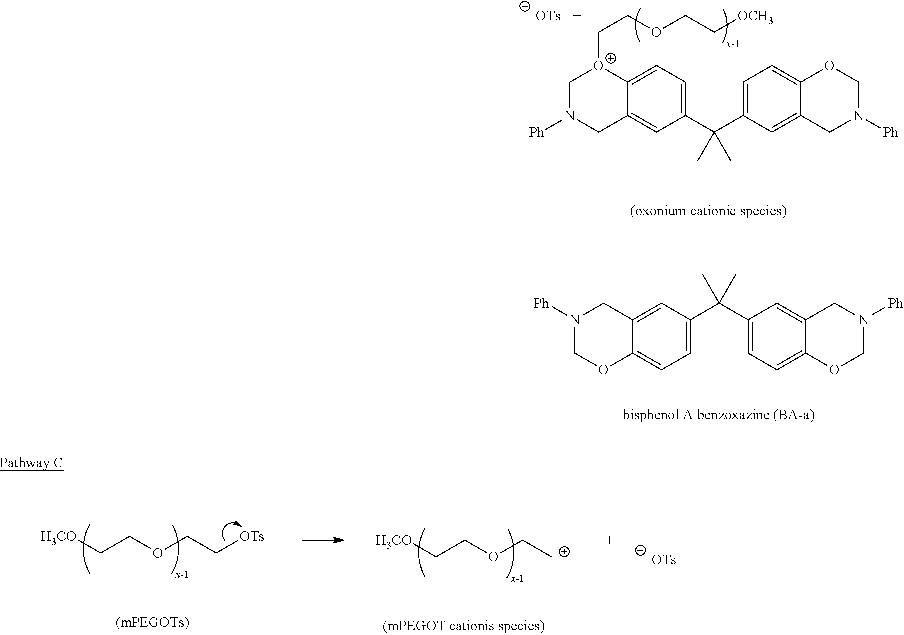

The strong nucleophilic and good leaving group characteristics of p-toluenesulfonic acid (HOTs) HOTs led to monomer conversion and reduced curing temperatures. Any HOTs generated in the blends from the mPEGOTs series could therefore act as an accelerant by analogous mechanisms. Alternative pathways that lead to cationic benzoxazinyl molecules to initiate the cationic polymerization of other neutral BA-a monomers are possible. Shown in Scheme 3 are these initiation and propagation steps. For clarity, the scheme first considers the conversion of one benzoxazinyl functional group from BA-a followed by a separated step for the cross-linking reaction for the other benzoxazinyl functional group. This proposed mechanism also applies the pegylated cationic oxonium species that was in common in both Pathway A and B in Scheme 2, and does not focus on other cationic species such as the iminium species or show the formation of N,O-acetal-type linkages that are also known for P(BA-a)s. After initiation and the generation of an initial flux of ⁻OTs, a free BA-a monomer attacks the cationic center of the oxonium initiator and the cationic charge is transferred to this attacking BA-a. Proton transfer restores the aromaticity in the attacking BA-a unit and leads to the Mannich bridged structure that is shown atop at the middle of Scheme 3. The process repeats itself with new BA-a monomer that adds to the cationic molecule so as to extend the polymer in a chain growth process. The ROP reaction of each benzoxazinyl functional group therefore leads to the more thermodynamically favored phenolic residue that should be further considered for reaction with mPEGOTs. These newly created phenol residues nucleophilic attack the tosylate end group of other mPEGOTs molecules and lead to random mPEG grafts along the PBA-a backbone with concomitant formation of HOTs, a byproduct associated with this propagation step. This secondary flux of HOTs was detected by both TGA-MS and TGA-FTIR. In the control series, the PBA-a was found to phase-separate from mPEGOH during the thermal ROP, and cured at higher temperatures. The pegylated oxonium initiator originating in the mPEGOTs blends would be expected to have high solubility in the BA-a/mPEGOTs miscible phase, which would allow the onset temperature for cure to be reduced relative to that of phase-separated analogues. The Gordon-Taylor-predicted T_{g} values for the cured BA-a/mPEGOTs blends and the FTIR evidence for strongly hydrogen bonded OH groups suggests that the grafting reaction of mPEG to the PBA-a proceeds to a large extent leading to a hybridized graft copolymer with a high compatibility with free mPEGOTs and/or BA-a.

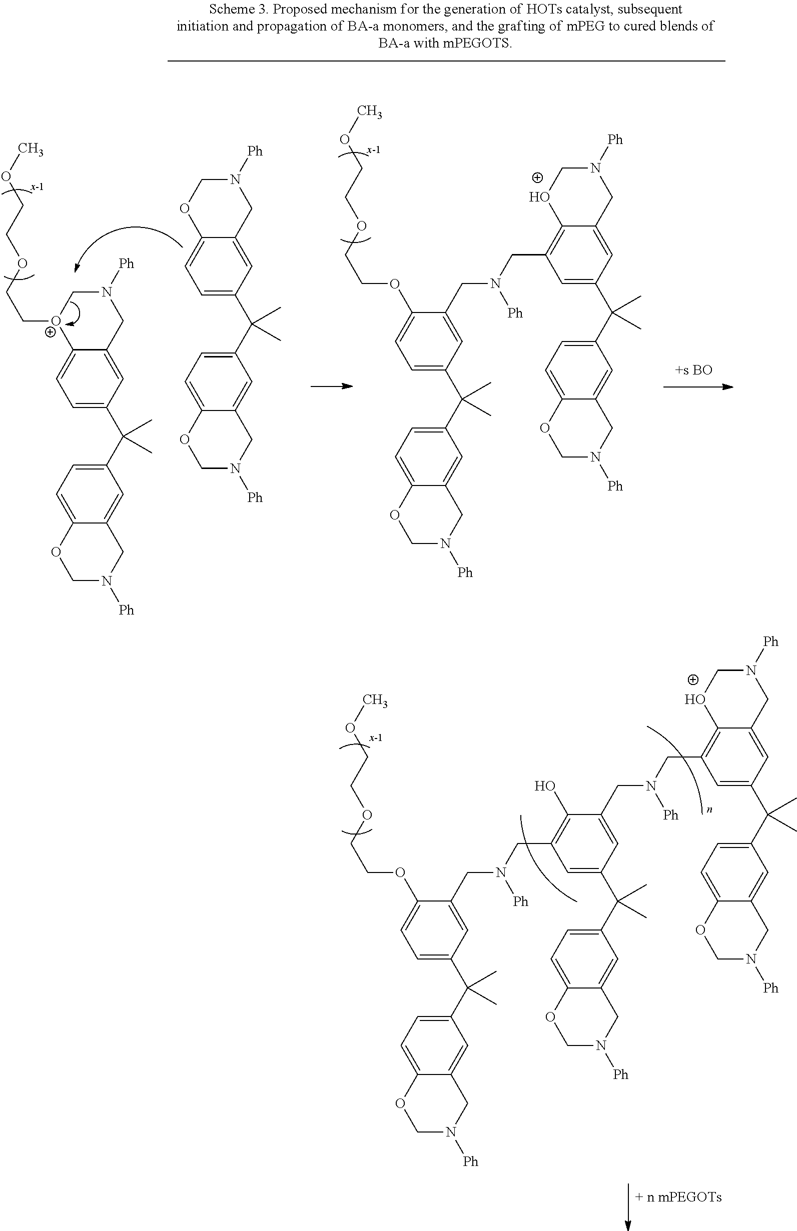

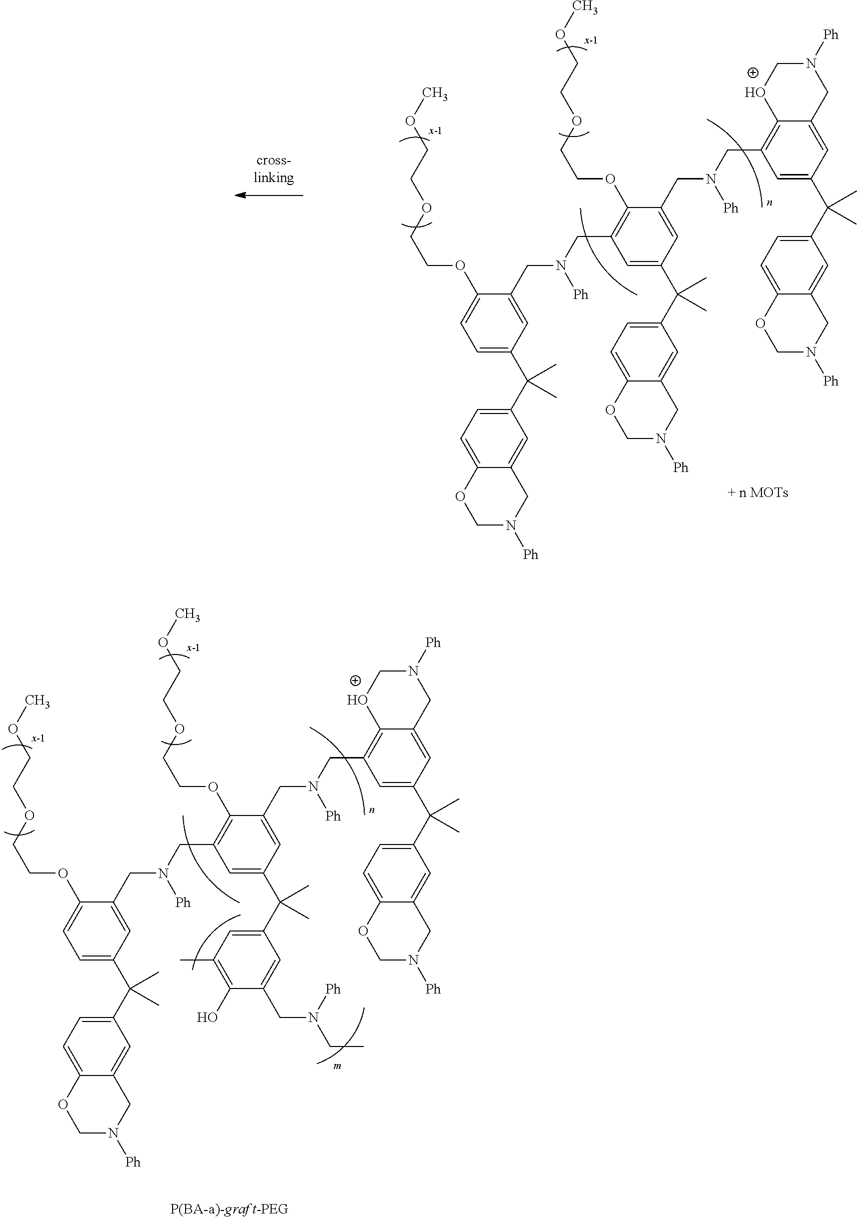

The roles of end-group functionalized methoxypolyethyleneglycol (mPEG) for solubilizing, accelerating, and grafting to a polybenzoxazine network derived from bis-phenol-A-based benzoxazine (BA-a) was studied. The nature of the end-group had little effect on the miscibility range for BA-a in a mPEG-based matrix, where a wide loading range of ˜100-40 wt. % BA-a was possible. When the end-group of the mPEG was a hydroxyl (OH) residue, the temperature required for ring-opening polymerization (ROP) of dissolved BA-a increased by ˜5-10° C. Conversely, when the end-group of the mPEG was a p-toluenesulfonate (OTs) residue, a significant decrease in temperature of the ROP was found with the onset for cure occurring at ˜20-60° C. less than that of the pure BA-a monomer. This onset for the cure temperature was linearly correlated to the weight loading of mPEGOTs in the blends and supports the assignment of the role of the mPEGOTs as an accelerant for BA-a cure. In-situ monitoring of the headspace above curing blends using FTIR and MS identified free tosylate and indicated that the mPEGOTs is activated by nucleophilic attack by BA-a at ˜110° C. in order to create HOTs catalysts and cationic initiators for the ROP of BA-a. The molecular structure of soluble extracts and the glass transition of cured mPEGOTs blends indicated that a significant amount of grafting has occurred in these materials, where, a phenolic rich structure including hydrogen-bonded phenols was present. The cured mPEGOH blends were more phenoxy-rich and had insignificant hydrogen bonding in any phenols. All cured mPEGOTs/PBA-a materials were transparent yellow-orange materials while those of cured mPEGOH/PBA-a were orange and opaque. A homogeneous P(BA-a)-graft-PEG molecular structure was assigned for the former whereas a phase-separated PBA-a/PEG structure was assigned for the latter. Scanning electron microscopy supported this assignment where a highly voided PBA-a resulted from solvent extraction of PEO from the phase-separated cured mPEGOH/PBA-a materials.

The resulting P(BA-a)-graft-PEG exhibited improved and tunable thermal and physical properties. The glass transition of the P(BA-a)-graft-PEG could be adjusted over a wide temperature range (40-160° C. experimentally demonstrated) where values followed a Gordon-Taylor relation based on the composition of the initial mPEGOTs/BA-a blend. Most P(BA-a)-graft-PEG exhibited higher thermal stability with a temperature of 276° C. (based on a 5 wt. % mass loss T_{D5%} metric) where most samples were more stable than pure PBA-a (T_{D5%}=214° C.) or materials created from phase-separated mPEGOH/PBA-a (T_{D5%}=202-225° C.).

TABLE 1
Equilibrium melting points for BA-a/mPEGOH and BA-a/mPEGOTs blends.

| | BA-a/ | BA-a/ | BA-a/ |
|---|---|---|---|
| BA-a:PEG | mPEGOH₂₀₀₀ | mPEGOTs₂₀₀₀ | mPEGOTs₉₀₀ |
| ratio | (° C.) | (° C.) | (° C.) |
| 0:100 | 50.79 | 50.1 | 24.77 |
| 10:90 | 50.34 | 47.86 | 25.56 |
| 0:80 | 47.06 | 45.81 | 24.56 |
| 30:70 | 46.18 | 45.14 | 22.99 |
| 40:60 | 45.77 | 39.27 | 21.23 |
| 50:50 | 41.92 | 38.93 | None |
| 60:40 | 42.67 | 38.68 | 22 |
| 80:20 | None | None | None |
| 100:0 | None | None | None |

TABLE 2
Enthalpy of benzoxazine cure exotherms for
BA-a/mPEGOH and BA-a/mPEGOTs blends.

| | BA-a/ | BA-a/ | BA-a/ |
|---|---|---|---|
| BA-a:PEG | mPEGOH₂₀₀₀ | mPEGOTs₂₀₀₀ | mPEGOTs₉₀₀ |
| ratio | (J/g) | (J/g) | (J/g) |
| 0:100 | 0 | 0 | 0 |
| 10:90 | 26.89 | 50.52 | — |
| 0:80 | 33.01 | 76.84 | 91.35 |
| 30:70 | 81.85 | 89.91 | — |
| 40:60 | 94.84 | 142.1 | 195.7 |
| 50:50 | 146.25 | 165.8 | — |
| 60:40 | 132.03 | 185.3 | 206.9 |
| 80:20 | 144.92 | 255.7 | — |
| 100:0 | 242.18 | 286.8 | 286.8 |

TABLE 3
Enthalpy of melting for PEG in BA-a/mPEGOH, BA-a/mPEGOTs,
PBA-a/mPEGOH and PBA-a/mPEGOTs blends.

| | Heat Cycle 1 | | Heat Cycle 2 | |
|---|---|---|---|---|
| | BA-a/ | BA-a/ | PBA-a/ | PBA-a/ |
| BA-a:PEG | mPEGOH-₂₀₀₀ | mPEGOTs₂₀₀₀ | mPEGOH₂₀₀₀ | mPEGOTs₂₀₀₀ |
| ratio | (J/g) | (J/g) | (J/g) | (J/g) |
| 0:100 | 148.8 | 164.8 | 142.2 | 105.9 |
| 10:90 | 125.5 | 134.2 | 125.2 | 106.4 |
| 20:80 | 95.32 | 116 | 98.7 | 103.6 |
| 30:70 | 98.23 | 103 | 92.88 | 88.82 |
| 40:60 | 86.05 | 74.55 | 75.26 | 18.89 |
| 50:50 | 59.12 | 48.05 | 55.55 | 0 |
| 60:40 | 9.082 | 3.188 | 43.52 | 0 |
| 80:20 | 0 | 0 | 0 | 0 |
| 100:0 | 0 | 0 | 0 | 0 |

### Example 2. High Resilience Thin Films from Polymerization of Blends of Benzoxazine and Tosylated-Polysulfone

Thin films of polybenzoxazine are attractive for many applications since these coatings can be stable to oxidizing environments, harsh chemicals, and high temperature conditions. Realizing such thin films is challenging due to the poor solubility of the polymer and since many benzoxazine monomers have poor film-forming properties owing to their low molecular weights and low viscosity in the melt state.

Polysulfones are an attractive polymer to select for creating robust thin films. Without wishing to be bound by theory, it is believed that polysulfone films can have a high resistance to physical impact and radiation, high mechanical and electrical stability over a wide temperature range (170° C.-230° C.), and can be compatible with steam sterilization. Polysulfones can be used, for example, for the manufacture of medical components, water filters, fuel cell membranes, and 3D-printing filaments.

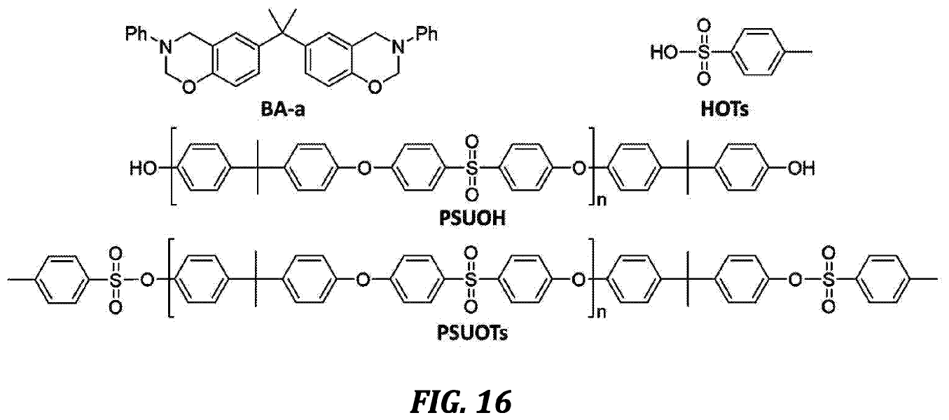

Induction heating is a fast and selective method for heating, melting, and welding various materials. In induction heating, an electromagnet is programmed to pass a high-frequency alternating current that generates a rapidly alternating magnetic field which penetrates a conducting material. The resulting eddy currents in the conductor generate heat by resistive heating. The use of induction heat is attractive for polymerization since it is rapid and efficient and operates with temporal control while being environmentally friendly. The solubility, polymerization and the molecular characterization of blends of benzoxazine in end-group tosylated polyethersulfone [α,ω-PSU(OTs)₂] were studied. Upon either radiative or induction heating, the films underwent a thermal ROP of included BPA-based benzoxazine (BA-a) and, in the case of α,ω-PSU(OTs)₂ films, afforded polybenzoxazine derivatives that were further covalently cross-linked through the PSU. Contrary to the end-group hydroxylated polyethersulfone blends, α,ω-PSU(OH)₂, a decrease in the cure temperature was found for the BA-a/α,ω-PSU(OTs)₂ combination. Cured thin films of BA-a/α,ω-PSU(OTs)₂ were found to have higher stiffness and higher resilience than that of the parent α,ω-PSU(OH)₂ and α,ω-PSU(OTs)₂ materials. In addition, the consistency of the mechanical properties in cured thin films of BA-a/α,ω-PSU(OTs)₂ was much higher than the BA-a/α,ω-PSU(OTs)₂ counterparts. The structures of BA-a, HOTs, PSUGH, and PSUOTs are shown in FIG. 16.

N-phenyl bisphenol-A benzoxazine (BA-a) monomer was used as received from Huntzman. End-group hydroxylated polyethersulfone, α,ω-PSU(OH)₂, (number average molecular weight, Mₙ=22,000 Da) and triethylamine (≥99% purity) were used as acquired from Sigma Aldrich. Methylene chloride (99.9% purity) and methanol (99.9%) was used as received from Fisher Scientific. Chloroform (99.8% purity) was used as received from J.T. Baker. Silica gel was used as received from Silicycle.

A Vega TS 5136MM scanning electron microscope (SEM), operated with an accelerating voltage of 15 kV, was used to capture micrographs of samples that were coated with a thin layer of gold and platinum to dissipate charge (nominal Au thickness ˜1 nm). Cured blend samples were prepared for SEM by fracturing the sample followed by a soak in chloroform for 30 minutes before drying in ambient conditions. A Nicolet iS10 FTIR spectrometer equipped with an attenuated total reflectance (ATR) accessory was used for FTIR studies (64 scans at 4 cm¹ resolution per spectra). A Bruker AVANCE-III, HD 500 MHz NMR spectrometer was used to collect H-NMR spectra. Atomic Force Microscopy images were taken with a Bruker Bioscope Catalyst in tapping mode. Conical shaped silicon tips were used that had cantilevers with a force constant of 40 N/m.

Preparation of End-Group Tosylated Polyethersulfone [α,ω-PSU(OTs)₂]. To a 100 mL reaction round bottom flask with a gas inlet, α,ω-PSU(OTs)₂ (Mn 22000 Da; 5.00 g, 0.227 mmol) was added and stirred in chloroform (23 mL) until completely dissolved. Triethylamine (1.82 mL, 13.0 mmol) was added to the stirred polymer solution under inert atmosphere. In a separate flask, p-toluenesulfonyl chloride (0.869 g, 4.56 mmol, 20 equiv.) was dissolved in chloroform (4.5 mL). This solution was then added dropwise to the reaction flask and allowed to stir at room temperature for 16 h under inert gas. The reaction mixture was precipitated in methanol and dried in a vacuum oven at room temperature for 12 hours. To remove the excess p-toluenesulfonic acid, the crude polymer product was dissolved in chloroform (25 mL) and passed through a silica column followed by elution with chloroform. The eluent was concentrated in vacuo yielding a white transparent solid (3.23 g). ¹H NMR (500 MHz, CDCl₃, ppm): δ 7.84 (d, main chain ArH), 7.80 (d, 2H, end group ArH), 7.35 (d, 2H, end group ArH), 7.23 (d, main chain ArH), 7.00 (d, main chain ArH), 6.93 (d, main chain ArH), 2.45 (s, end group CH₃), 1.69 (s, CH).

Preparation of Blends of Polyethersulfone and N-Phenyl Bisphenol A Benzoxazine (BA-a). The general procedure for the preparation of blends of BA-a with α,ω-PSU(OH)₂ or α,ω-PSU(OTs)₂ is as follows. Appropriate amounts of BA-a and PSU were measured into a 10 mL round bottom flask and dissolved in 5 ml chloroform. Each sample was dried with a rotary evaporator and then dried in a vacuum oven at room temperature for 12 hours.

Thermal Analysis. All weight changes and char yield studies were conducted with a TA Instruments Q500 thermogravimetric analyzer. The pre-cured samples were held at an isotherm for 5 minutes at 30° C. and then ramped up 10° C. per minute from 30 to 900° C.

A TA Instruments Q100 differential scanning calorimeter was used for all cure and glass transition studies. All samples were scanned at a rate of 5° C. per minute with an amplitude of ±0.531° C. in a heat-cool-heat run from 30° C. to 260° C. and back to 30° C. A tangential sigmoidal baseline was used to estimate the exothermic cure by integrating the peak. The inflection point of the step change was used to calculate the glass transitions of all the samples.

Preparation of Freestanding Films. A Laurell ws-400b-6npp/lite spin coater was used to prepare the thin films. PSU/BA-a blends were dissolved in chloroform to make a solution with a polymer concentration of ˜100 mg/ml. ˜0.4 mL of the solution is pipetted onto a glass substrate (˜57 mm²) and spin-coated at 2000 rpm for 60 sec with an acceleration of 2650 rpm/s. The spin coated samples are then placed in a vacuum oven programed to ramp from 25° C. to 225° C. for one hour and hold heat at 225° C. for another hour. At this point, samples are removed and cooled to room temperature in ambient atmosphere. A doctor blade is used to score the edges of the film from the glass substrate edge and the sample is placed into an ultra-pure water bath for 30 min to relieve the thin film from the substrate. A support frame made from Kapton tape is used to capture the floating film from the air-water interface and this is dried at ambient conditions for a minimum of 2 h prior to test work.

Mechanical analysis. A minimum of three stress-strain curves of each PSU/PBA-a film was obtained with an ADMET load cell with a cross-head speed of 0.55 mm/min.

Preparation and Characterization of α,ω-PSU(OH)₂ or α,ω-PSU(OTs)₂. The two PSU materials used to study the formation and properties of cross-linked thin films containing polybenzoxazine differed by their end-group. The transformation of the α,ω-PSU(OH)₂ into α,ω-PSU(OTs)₂ is outlined in Scheme 4. The structure and molecular weight of the α,ω-PSU(OH)₂ was confirmed by H-NMR and gel permeation chromatography. The hydroxyl end groups of the polymer were converted into p-toluenesulfonyl (tosylate) leaving groups using p-toluene sulfonyl chloride. For α,ω-PSU(OH)₂, the ¹H-NMR spectra agreed with previous reports with a signal at 7.35 ppm for the aromatic BPA-based end-groups and a separate signal at 1.69 ppm for the dimethyl functionality of repeat unit BPA groups. The respective integral ratio of the two was 180:1.92 which indicated an average degree of polymerization of 60 and therefore a Mₙ of 26.5 kDa. The ¹H-NMR spectra for the resulting α,ω-PSU(OTs)₂ evidenced new aromatic signals at 7.80 (J=0.0137 ppm) ppm and 7.35 ppm (J=0.0161 ppm) and a new methyl singlet for the tolyl functionality at 2.45 ppm. The ratio of the integral of the singlet at 2.45 ppm to the doublet at 7.35 ppm was 3:2.1, a value close to the theoretical value of 3:2. The ratio of the integral of the singlet at 2.45 ppm to the singlet for the BPA-based repeat of PSU at 1.69 ppm was 6:180, indicating an average degree of polymerization of 60 and therefore a Mₙ of 26.5 kDa (based on a repeat unit MW of 442.5 Da). The ratio of the integral of the multiplet at 7.68 ppm to the singlet for the BPA-based repeat of PSU at 1.69 ppm was 2.03:180, indicating an average degree of polymerization of 60 and therefore a Mₙ of 26.5 kDa. The comparison of the GPC traces for the α,ω-PSU(OH)₂ and α,ω-PSU(OTs)₂ showed that both had a monomodal molecular weight profile and similar molecular weight distribution, signifying that the tosylation reaction conditions were very specific to conversion of the polymer end-groups and do not favor any side-reactions on the rest of the polymer structure.

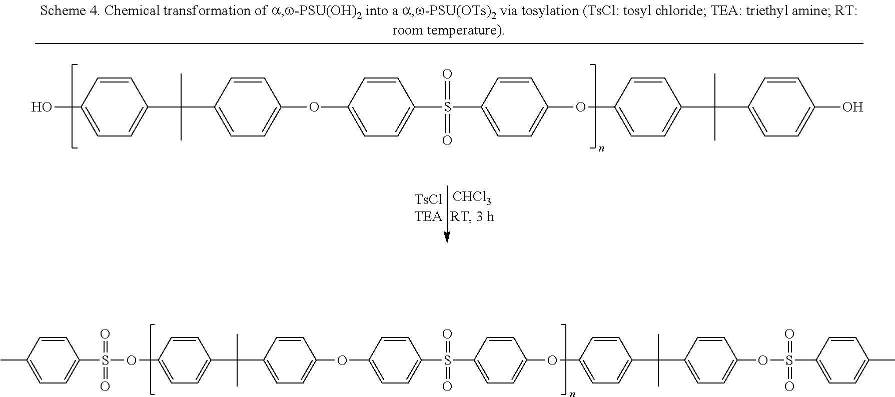

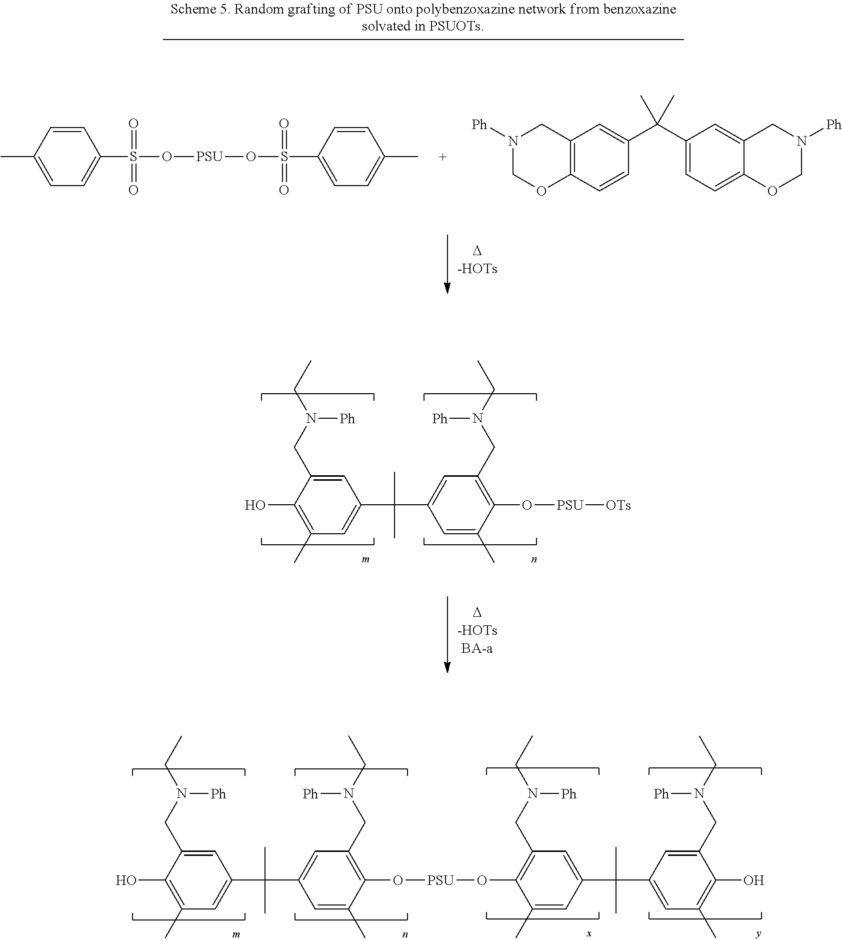

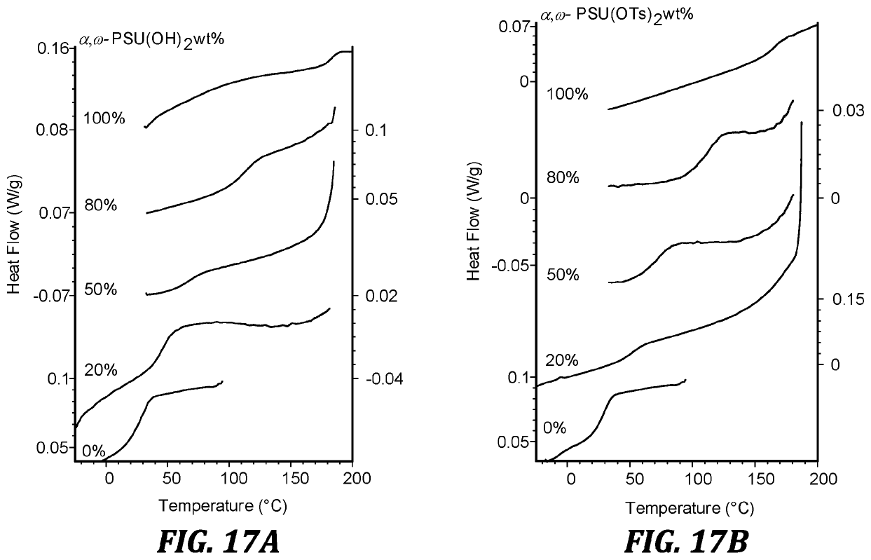

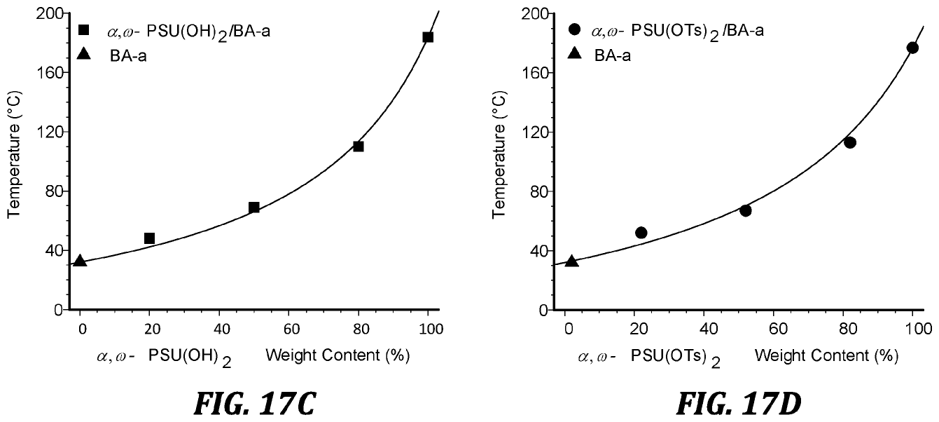

Miscibility of in PSU/BA-a Blends. Solvent-free PSU/BA-a blends were studied by differential scanning calorimetry (DSC). A heat-cool-heat program was selected to characterize the thermal transitions of the PSU in blends prior to thermal ROP of BA-a, the cure BA-a monomers and any possible reaction between the resulting PBA-a with the PSU. Mixtures of BA-a with either α,ω-PSU(OH)₂ and α,ω-PSU(OTs)₂ were prepared by dissolving an appropriate mass of each in chloroform, a common solvent for all materials, followed by rapid evaporation of the solvent. No thermal quenching of the blends was conducted since the PSU has a high glass transition temperature (T_{g}=175-185° C.). FIG. 17A shows the DSC traces for the first heating curve for α,ω-PSU(OH)₂/BA-a blends and the pure BA-a and α,ω-PSU(OH)₂. FIG. 17B shows the DSC traces for the first heating curve for α,ω-PSU(OTs)₂/BA-a blends and the pure BA-a and α,ω-PSU(OTs)₂. The topmost traces in FIGS. 17A and 17B show data for the pure α,ω-PSU(OH)₂ and α,ω-PSU(OTs)₂, respectively. The traces revealed weak T_{g} signals for α,ω-PSU(OH)₂ and α,ω-PSU(OTs)₂ at 187° C. and 177° C., respectively. The bottommost traces in FIGS. 17A and 17B report the same dataset for the first heating curve for pure BA-a and reveal a T_{g} for the monomer at 32° C. The T_{g} values found for all of binary mixtures of PSU with BA-a were similar between the two types of blends. Shown in FIGS. 17C and 17D are these T_{g} values plotted versus the nominal PSU content in the blends. All blends exhibited a T_{g} above 35° C., which indicated that thin films or coatings would have stability for room temperature processing. Assuming that the blends remain amorphous and that they exhibit an ideal volume of mixing and additivity, a Gordon-Taylor relation can be applied to these plots. The fitting constants, k, for the curves in FIGS. 17C and 17D are statistically similar with values of 0.29±0.03 and 0.33±0.03, respectively, and indicate that there is good miscibility as a result of favorable secondary bonding interactions between the BA-a and the PSU polymer in both series.

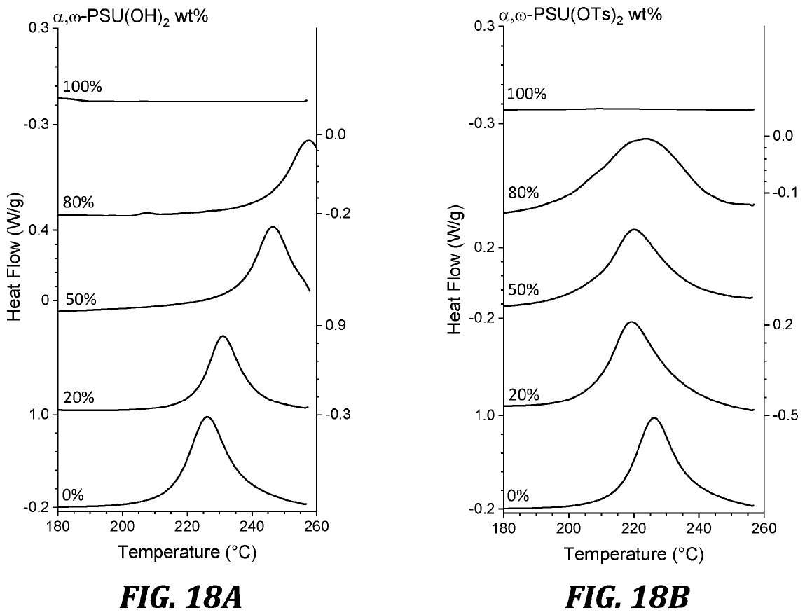

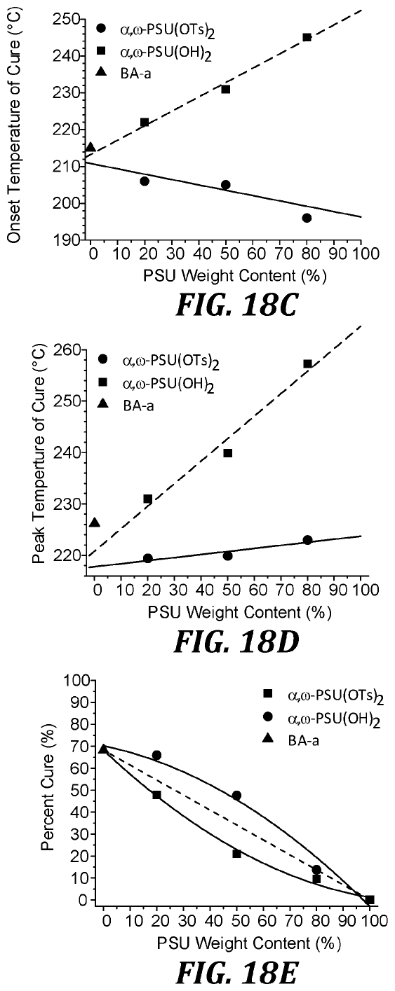

Thermal ROP of BA-a in α,ω-PSU(OH)₂ and α,ω-PSU(OTs)₂ blends. Ring opening polymerization (ROP) of BA-a occurs at elevated temperatures (>160° C.) and proceeds via cationic polymerization. The DSC study was extended to include a temperature range for the cure of the dissolved monomer in the PSU blends. FIG. 18A shows the DSC trace for pure BA-a, which depicted an exotherm with a peak value and an onset value of 226 and 215° C., respectively. The area of the exotherm corresponded to the enthalpy of thermal ROP of BA-a and had a value of 241 J/g. As PSU is introduced in the α,ω-PSU(OH)₂ blend series (up the stack plot in FIG. 18A), the exothermic ROP signals remained symmetric and approximately monomodal and were found to reduce in enthalpy and to shift to higher temperatures with peak values in the range of 230-255° C. The analogous data for the α,ω-PSU(OTs)₂ series depicted important distinctions. In this series, the thermal ROP exotherms shifted to a lower temperature than that for the pure BA-a material. All exotherms in this series exhibited peak temperatures in the range of 218-222° C. Shown in FIGS. 18C and 18D are the plots for the onset and peak temperature values the thermal ROP exotherm versus the nominal weight percent of α,ω-PSU(OH)₂ and α,ω-PSU(OTs)₂, respectively. The cure temperature of BA-a increased in a near-linear fashion as the weigh fraction of the in α,ω-PSU(OH)₂ increased. The increasing cure temperatures suggested that the steps for initiation and propagation were not accelerated by interactions between BA-a with PSU repeat units, or the hydroxyl end groups of the polymer, but were limited by kinetic barriers such as reduced heat transfer and higher viscosity in the polymer blends. The corresponding data for the α,ω-PSU(OTs)₂ series showed that the onset and peak temperatures of the thermal ROP exotherm for blended BA-a were less than that of the pure BA-a. Therefore, the tosylate end-groups of the α,ω-PSU(OTs)₂ acted as cure accelerants from the thermal ROP of BA-a. The enthalpy values for the exotherm were also found to be less than that from the thermal ROP of pure BA-a. To further analyze the enthalpy of polymerization in each series, the enthalpy values were weighted relative to the fractional amount of BA-a in each blend and normalized to the standard enthalpy of polymerization of 354 J/g. These data, reported as the percent cure of BA-a, were co-plotted against the theoretical cure for a fractional BA-a blend represented by the dotted trace in FIG. 18E. The percent cure values of BA-a in the α,ω-PSU(OH)₂ series were all found to be less than the theoretical curve. Conversely, the percent cure values of BA-a in the α,ω-PSU(OTs)₂ series were all larger than the theoretical prediction. Without wishing to be bound by theory, it is believed that this excess enthalpy as energy released is due to grafting of the tosylated polymer to the P(BA-a) network.

DSC traces at a high temperature range (180-260° C.) depicted an exothermic peak corresponding to the energy released from the ROP of BA-a. The onset, peak temperature, and enthalpy of polymerization are 215° C., 226° C., and 68 J/g, respectively. The exothermic cure peak was present in all blend samples and shifted to increasing temperature values in the α,ω-PSU(OH)₂/BA-a blend series. Monomodal cure exotherm peak in these PSU series were ascribed a secondary mode in the cure exotherm as one due to phase separation arising from crystallization of the PEG polymer. The BA-a monomer was therefore uniformly dispersed in the PSU matrix.

TABLE 4
Onset, cure temperature, before cure T_{g}, after cure T_{g}, and cure enthalpy of
blend type a) an OTs (tosylate) system; and blend type b) an OH (hydroxyl) system.

| a) | | | | Before | After |
|---|---|---|---|---|---|
| Blend type | Onset | Cure | Cure | cure | Cure |
| α,ω- | Temp | Temp | Enthalpy | Tg | Tg |
| PSU(OTs)2/BA-a | ° C. | ° C. | J/g | ° C. | ° C. |
| 100/0 | — | — | — | 180 | 180 |
| 90/10 | — | — | — | | 184 |
| 80/20 | 196 | 222 | 47 | 113 | 181 |
| 50/50 | 205 | 220 | 168 | 67 | 172 |
| 20/80 | 206 | 219 | 233 | 52 | 167 |
| 0/100 | 215 | 226 | 241 | 32 | 160 |
| b) | | | | Before | After |
| Blend type | Onset | Cure | Cure | cure | Cure |
| α,ω- | Temp | Temp | Enthalpy | Tg | Tg |
| PSU(OH)2/BA-a | ° C. | ° C. | J/g | ° C. | ° C. |
| 100/0 | — | — | — | 187 | 187 |
| 90/10 | — | — | — | | 184 |
| 80/20 | 245 | 257 | 34 | 110 | 161 |
| 50/50 | 231 | 240 | 73 | 69 | 160 |
| 20/80 | 222 | 231 | 169 | 48 | 173 |
| 0/100 | 215 | 226 | 241 | 32 | 160 |

The thermal ROP parameters found by DSC curves for blends from the α,ω-PSU(OTs)₂ series have important distinctions. For comparison, in the DSC trace of the pure BA-a sample (FIG. 18B), as α,ω-PSU(OTs)₂ is increased in this blend series, the onset and peak temperatures of the exotherm of the thermal ROP reaction for BA-a decreased to lower values. These exotherms signals were also symmetric and monomodal confirming a similar level of mixing to the α,ω-PSU(OH)₂ system. The temperature of the peak exotherm was ˜5-10° C. lower than that of the parent BA-a data set while the onset values decreased with a roughly linear trend line. For the most α,ω-PSU(OTs)₂ rich sample, the onset value was ˜20° C. lower than that of the parent BA-a. The decrease in the cure conditions confirmed that the tosylate end groups function as cure accelerants for the thermal ROP of BA-a. The decrease in cure in the α,ω-PSU(OTs)₂ system was significant when compared to the mPEGOTs system because the concentration of tosyl endgroups vary greatly. The PSU system had an endgroup concentration of 77 μmol/g whereas the mPEG system had an end group concentration of 500 μmol/g. In spite of the low concentration of tosylate end groups, a significant decrease in cure temperature was found.

The Gordon Taylor relationship (see Example 1) can be used create a relationship that quantifies the miscibility between varying ratios of a mixture of two molecules. The constant k is used to fit the data to determine the tunability of the glass transition of the polymer alloy. A k value near 1 indicates perfect miscibility whereas a k value of 0 indicates phase separation.

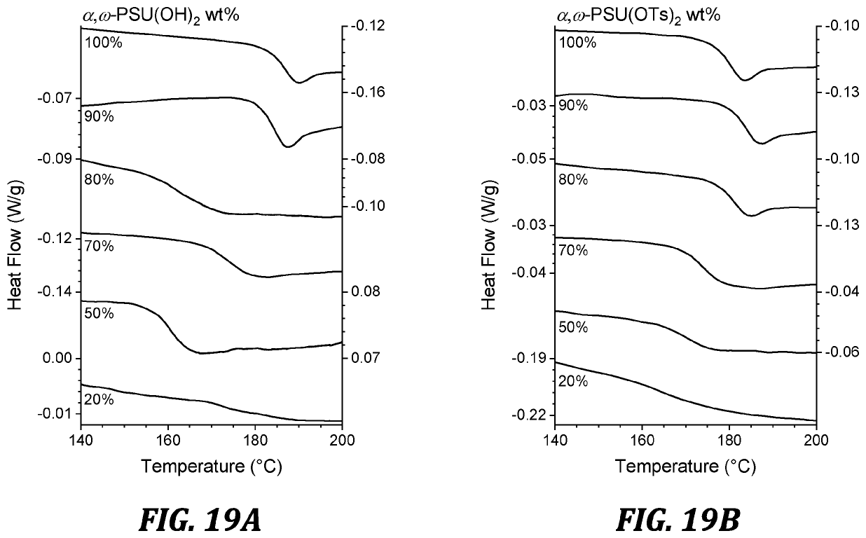

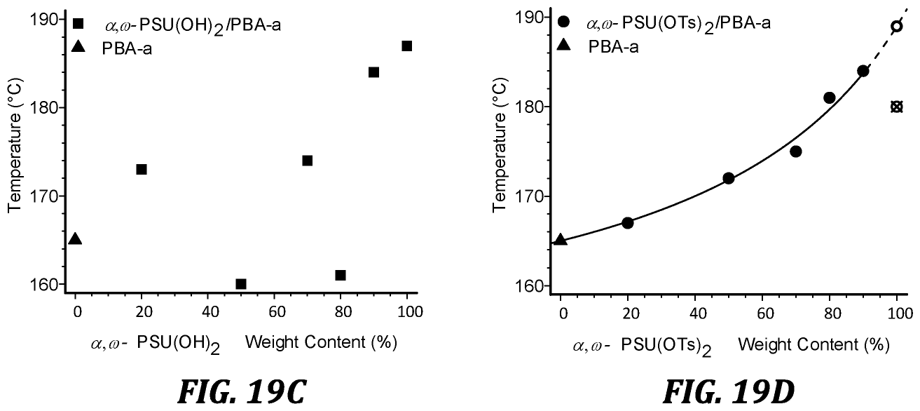

Both types of blends were miscible before cure. After cure, referring to FIGS. 19A-19D, there was evident phase separation in the α,ω-PSU(OH)₂ blends as there was no obvious Gordon Taylor trend present. The mixing in the α,ω-PSU(OTs)₂ blends increased after cure, as evidenced from the increased k value after cure. The T_{g} of 189° C. of the 90% α,ω-PSU(OTs)₂ blend was greater than the T_{g} of 184° C. of the neat α,ω-PSU(OTs)₂.

FTIR spectra of cured blends of PBA-a with PSUGH and PSUOTs were carried out and were indicative of full polymerization of the PBA-a.

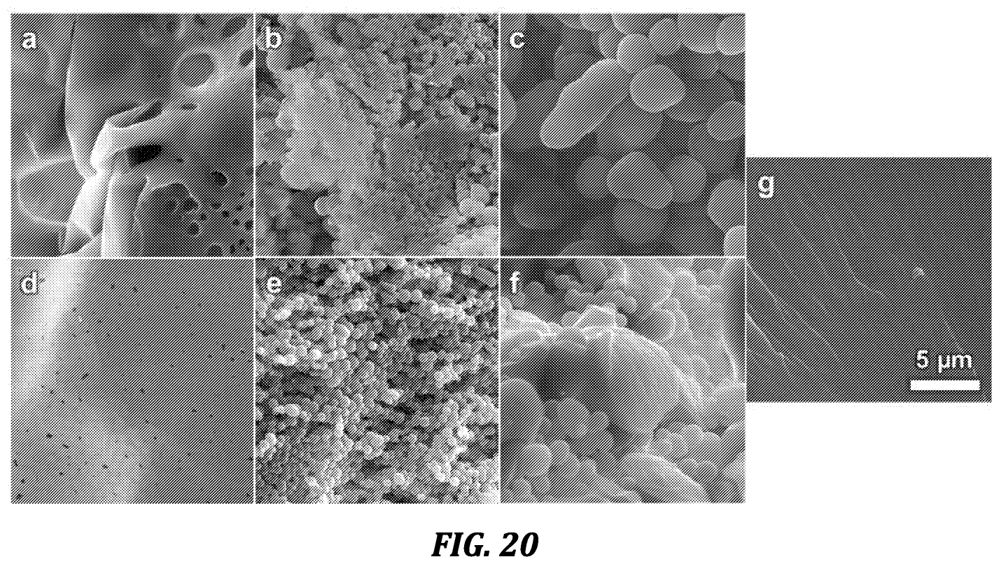

Referring to FIG. 20, the SEM images revealed very different morphologies of the fractured surfaces of the α,ω-PSU(OH)2 and α,ω-PSU(OTs)2 blends. FIG. 20(g) shows the smooth morphology of pure cured benzoxazine. Most of the images of the blends had a sphere like morphology. It is believed that aromatic polymers can form micro pockets in which benzoxazine can be cured in. As the benzoxazine cures, the two polymers are driven to phase separation. The consistent dispersion and uniformity observed in the OTs blends are believed to be due to the miscibility of the blends. FIG. 20(e) has high uniformity in its morphology that indicate high uniformity in nucleation and polymerization. It is believed that the grafting prevented phase separation and allowed α,ω-PSU(OTs)2 to envelop the BA-a in micro pockets. The inconsistent and amorphous spheres seen in the OH blends can be due to the mid cure phase separation that opened the microspheres and allowed the benzoxazine spheres to deform. FIG. 20(b) shows more of an amorphous morphology indicating less uniformity in the nucleation and polymerization of the blend. This may be due to the competing (and increasing) phase separation over the course of the cure. The film like structure FIG. 20(a) may be caused by a lack of polymer to envelop all of the BA-a during polymerization of benzoxazine. There are large voids present in the 80% α,ω-PSU(OH) blend where the phase separated polysulfone dissolved out during the extraction wash. There are two types of morphologies visible in FIG. 20(f). There are the spheres and the branched network. It is believed that the spheres are pure benzoxazine and that the branched network is polysulfone grafted onto benzoxazine.

Thin films were prepared the same way for AFM imaging as they were for the stress-strain experiments. They were not lifted off the glass substrate for imaging. The AFM images were analogous to SEM images shown in FIG. 20. Specifically, the α,ω-PSU(OTs)₂ blends had a uniform spherical dispersion across the images that corresponded with the homogenous spheres seen in the SEM images. Random spheres in the α,ω-PSU(OH)₂ blends line up with the amorphous morphology seen in the analogous SEM images.

### Mechanical Analysis.

An ADMET load cell was used to obtain stress strain curves of the PSU/BA-a thin films. A cross-head speed of 0.55 mm/min was used and at least three trials were conducted on each type of film.

The tensile modulus of the α,ω-PSU(OTs)₂ blends consistently had a 95% confidence interval of no more than 3 Mpa. The 80:20 α,ω-PSU(OTs)₂:BA-a blend has a statistically higher tensile modulus than neat α,ω-PSU(OTs)₂. The α,ω-PSU(OH)₂ blends have very large 95% confidence intervals, making them unreliable in practical uses.

The resilience of the α,ω-PSU(OTs)₂ blends were statistically consistent. The resilience of the α,ω-PSU(OH)₂ blends decreased in a linear fashion until the 80:20 blend. The 80:20 α,ω-PSU(OTs)₂:BA-a blend was more resilient than the α,ω-PSU(OH)₂ blends on a 95% confidence interval.

Cured thin films of BA-a/α,ω-PSU(OTs)₂ were found to have 28% high modulus than that of the parent α,ω-PSU(OH)₂ and α,ω-PSU(OTs)₂ materials. In addition, the consistency of the mechanical properties in cured thin films of BA-a/α,ω-PSU(OTs)₂ was much higher than the BA-a/α,ω-PSU(OTs)₂ counterparts, with films from the former series exhibiting a relative standard deviation of only 5.5% compared to 60% for the latter.

While illustrative embodiments have been illustrated and described, it will be appreciated that various changes can be made therein without departing from the spirit and scope of the disclosure.

1. A polybenzoxazine-containing composition, comprising:
a polybenzoxazine, and
a covalently-grafted polymer on the polybenzoxazine, a covalently-grafted particle on the polybenzoxazine, a covalently-grafted fiber on the polybenzoxazine, or any combination thereof,
wherein the covalently-grafted polymer is different from the polybenzoxazine.

2. The polybenzoxazine-containing composition of claim 1, wherein the covalently-grafted polymer is independently selected from poly(ethylene glycol), polyethers, polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters, polyurethanes (PU), bimaleimide polymers (BMIs), fluorinated polymers, vinylics, conjugated polymers, copolymers thereof, and any combination thereof.

3. The polybenzoxazine-containing composition of claim 1, wherein the covalently-grafted polymer is independently selected from poly(ethylene glycol), polysulfone (PSU), polyvinyl alcohol, and any combination thereof.

4. (canceled)

5. The polybenzoxazine-containing composition of claim 1, wherein the particle is selected from talc particle, silica, silicate particle, zeolite particle, titania particle, and any combination thereof.

6. The polybenzoxazine-containing composition of claim 1, wherein the fiber is selected from carbon nanotubes, fiberglass, and any combination thereof.

7. The polybenzoxazine-containing composition of claim 1, wherein the particle and/or fiber is further functionalized with a polymer independently selected from poly(ethylene glycol), polyethers, polysulfones (PSU), polyethersulfone (PES), poly(arylene sulfone) (PAS), poly(aryl ether sulfone)s (PAES), polyphenyl ethers (PPEs), polyphenylene oxides (PPOs), polyamides (PA), polycarbonate (PC), polyesters, polyurethanes (PU), bimaleimide polymers (BMIs), fluorinated polymers, vinylics, conjugated polymers, and/or copolymers thereof.

8-10. (canceled)

11. The polymer-grafted polybenzoxazine of claim 1, wherein the polybenzoxazine is linear having two termini.

12. The polymer-grafted polybenzoxazine of claim 11, wherein the polymer is covalently grafted onto a terminus of the polybenzoxazine.

13. The polymer-grafted polybenzoxazine of claim 11, wherein the polymer is grafted onto both termini of a linear polybenzoxazine.

14. (canceled)

15. The polymer-grafted polybenzoxazine of claim 11, wherein the linear polybenzoxazine is crosslinked by the polymer to provide a network.

16. (canceled)

17. The polymer-grafted polybenzoxazine of claim 1, wherein the polybenzoxazine is branched, and wherein the branched polybenzoxazine is optionally crosslinked by the polymer to provide a network.

18. (canceled)

19. A method of making a polymer-grafted polybenzoxazine, comprising:
providing a blend of a benzoxazine-containing monomer and a polymer functionalized with at least one leaving group selected from perfluoroalkylsulfonates, mesylates, and halides; and
curing the blend at a predetermined temperature to provide the polymer-grafted polybenzoxazine of claim 1.

20. The method of claim 19, wherein the benzoxazine-containing monomer comprises a 1,3-benzoxazine, optionally substituted with 1, 2, 3, 4, or 5 substituents independently selected from halo, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₁₋₆ alkoxy, C₁₋₆ haloalkoxy, aryl, heteroaryl, cycloalkyl, heterocycloalkyl, CN, NO₂, and OH.

21-22. (canceled)

23. The method of claim 19, wherein the benzoxazine-containing monomer is selected from 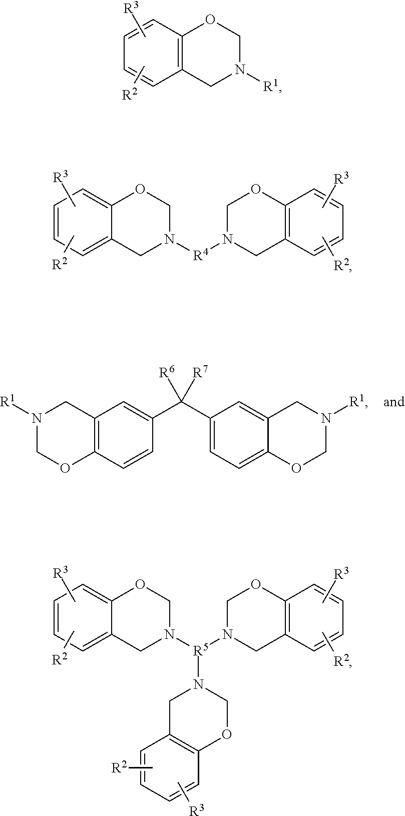
wherein:
R¹, R², R³ are each independently selected from halo, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₁₋₆ alkoxy, C₁₋₆ haloalkoxy, aryl, heteroaryl, cycloalkyl, heterocycloalkyl, CN, NO₂, and OH;
R⁴ is selected from alkenylene, arylene, haloalkenylene, cycloalkylene, heteroalkylene, and heteroarylene;
R⁵ is selected from alkenylene, arylene, haloalkenylene, cycloalkylene, heteroalkylene, and heteroarylene; and
R⁶ and R⁷ are selected from hydrogen, alkyl, C₁₋₆ haloalkyl, aryl, heteroaryl, cycloalkyl, heterocycloalkyl.

24-26. (canceled)

27. The method of claim 19, wherein the polymer is selected from poly(ethylene glycol), polysulfone, poly(phenylene oxide), poly(lactic acid), poly(glycolic acid), polyvinyl alcohol, a fluoropolymer, and any combination thereof.

28. The method of claim 19, wherein the at least one leaving group functionalizes a terminus of the polymer.

29. The method of claim 19, wherein the polymer functionalized with at least one leaving group is present in the blend at a mole % relative to the benzoxazine units of 0.01-90%.

30-36. (canceled)

37. A polybenzoxazine composite, comprising:
a polybenzoxazine, and
a particle and/or a fiber covalently grafted onto the polybenzoxazine.

38-52. (canceled)

53. A method of making the polybenzoxazine composite, comprising:
providing a blend of a benzoxazine-containing monomer and a particle and/or fiber functionalized with at least one leaving group selected from perfluoroalkylsulfonates, mesylates, and halides; and
curing the blend at a predetermined temperature to provide the polybenzoxazine composite of claim 37.

54-56. (canceled)

57. The method of claim 53, wherein the benzoxazine-containing monomer is selected from 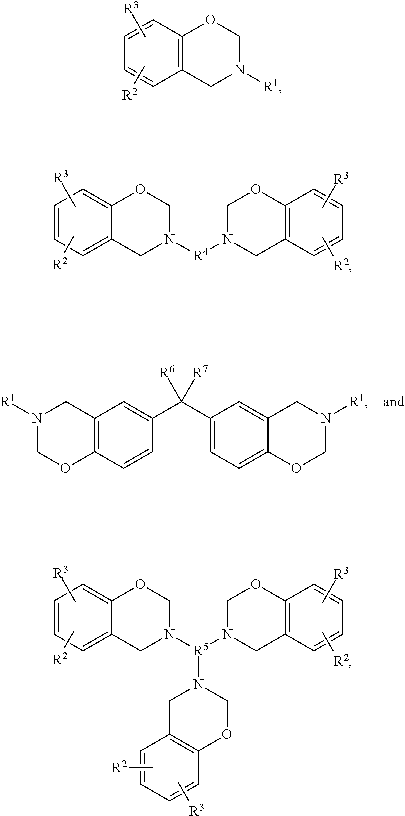
wherein:
R¹, R², R³ are each independently selected from halo, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₁₋₆ alkoxy, C₁₋₆ haloalkoxy, aryl, heteroaryl, cycloalkyl, heterocycloalkyl, CN, NO₂, and OH;
R⁴ is selected from alkenylene, arylene, haloalkenylene, cycloalkylene, heteroalkylene, and heteroarylene;
R⁵ is selected from alkenylene, arylene, haloalkenylene, cycloalkylene, heteroalkylene, and heteroarylene; and
R⁶ and R⁷ are selected from hydrogen, alkyl, C₁₋₆ haloalkyl, aryl, heteroaryl, cycloalkyl, heterocycloalkyl.

58-60. (canceled)

61. The method of claim 53, wherein the particle and/or fiber is further functionalized with a polymer selected from poly(ethylene glycol), polysulfone, poly(phenylene oxide), poly(lactic acid), poly(glycolic acid), a fluoropolymer, polyvinyl alcohol, and any combination thereof.

62-70. (canceled)
